(19) United States
(12) Patent Application Publication
(10) Pub. No.: US 2024/0277501 A1
(43) Pub. Date: Aug. 22, 2024

(54) FENESTRATED VASCULAR AORTIC REPAIR STENT, SYSTEMS, AND METHODS

(71) Applicant: Merit Medical Systems, Inc., South Jordan, UT (US)

(72) Inventors: Christopher Cindrich, Highland, UT (US); Joanne Hislop, American Fork, UT (US); Mattias Nielsen, North Salt Lake, UT (US)

(21) Appl. No.: 18/442,312

(22) Filed: Feb. 15, 2024

Related U.S. Application Data
(60) Provisional application No. 63/485,819, filed on Feb. 17, 2023.

(51) Int. Cl. A61F 2/954; A61F 2/06; A61F 2/966

(52) U.S. Cl. CPC A61F 2/954; A61F 2/966; A61F 2002/061; A61F 2002/067

(57) ABSTRACT

Endovascular prostheses used to treat diseased blood vessels, such as arteries, are disclosed. In some embodiments, an endovascular prosthesis is configured to be implanted within a diseased blood vessel adjacent a diseased section. The endovascular prosthesis may include a fenestration tube through which a guidewire extends in a sealed configuration. The fenestration tube can be selectively openable and configured to sealingly receive an expandable endovascular prosthesis that extends into a side branch vessel of the diseased blood vessel.

### RELATED APPLICATIONS

This application claims priority to U.S. Provisional Application No. 63/485,819, filed on Feb. 17, 2023 and titled, “Fenestrated Vascular Aortic Repair Stent, Systems, and Methods” which is hereby incorporated by reference in its entirety.

### TECHNICAL FIELD

The present disclosure relates to medical devices, including endovascular prostheses. In some embodiments, the present disclosure relates to a fenestrated endovascular prosthesis that provides access to branch arteries when implanted in a major artery, such as the aorta. Methods of manufacture, methods of use, and related systems (e.g., a deployment device for the fenestrated endovascular prosthesis, guidewire management systems, and the like) for the fenestrated endovascular prosthesis are also disclosed.

### BRIEF DESCRIPTION OF THE SEVERAL VIEWS OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an example of a fenestrated endovascular prosthesis.

FIG. 2A is a perspective view of an example of a fenestration tube of the fenestrated endovascular prosthesis of FIG. 1 in an open configuration.

FIG. 2B is a perspective view of an example of the fenestration tube of FIG. 2A of the fenestrated endovascular prosthesis of FIG. 1 in a sealed configuration.

FIG. 3A is a cross-sectional view of a fenestrated endovascular prosthesis-forming mandrel covered with a first material.

FIG. 3B is a cross-sectional view of the fenestrated endovascular prosthesis-forming mandrel of FIG. 3A covered with the first material and including fenestration tubes.

FIG. 3C is a cross-sectional view of the fenestrated endovascular prosthesis-forming mandrel of FIG. 3B covered with the first material, including the fenestration tubes, and being covered with a second material.

FIG. 3D is a cross-sectional view of the fenestrated endovascular prosthesis-forming mandrel of FIG. 3C covered with the first material, including the fenestration tubes, covered with the second material, and including a wire stent.

FIG. 4A is a side section view of the fenestrated endovascular prosthesis of FIGS. 1 and 3D with the fenestration tubes in the sealed configuration.

FIG. 4B is a top-down view of the fenestrated endovascular prosthesis of FIG. 4A along plane 4B-4B of FIG. 4A with the fenestration tubes in the closed configuration.

FIG. 4C is a bottom-up view of the fenestrated endovascular prosthesis of FIG. 4A along plane 4C-4C of FIG. 4A with the fenestration tubes in the closed configuration.

FIG. 5A is a side section view of the fenestrated endovascular prosthesis of FIG. 4A with guidewires extending through the fenestration tubes.

FIG. 5B is a top-down view of the fenestrated endovascular prosthesis of FIG. 5A along plane 5B-5B of FIG. 5A with guidewires extending through the fenestration tubes.

FIG. 5C is a bottom-up view of the fenestrated endovascular prosthesis of FIG. 5A along plane 5C-5C of FIG. 5A with guidewires extending through the fenestration tubes.

FIG. 6A is a side section view of the fenestrated endovascular prosthesis of FIG. 5A with fenestration dilators extending along the guidewires and through the fenestration tubes.

FIG. 6B is a top-down view of the fenestrated endovascular prosthesis of FIG. 6A along plane 6B-6B of FIG. 6A with the fenestration dilators extending along the guidewires and through the fenestration tubes.

FIG. 6C is a bottom-up view of the fenestrated endovascular prosthesis of FIG. 6A along plane 6C-6C of FIG. 6A with the fenestration dilators extending along the guidewires and through the fenestration tubes.

FIG. 7A is a side section view of the fenestrated endovascular prosthesis of FIG. 6A with secondary endovascular prostheses deployed in the fenestration tubes.

FIG. 7B is a top-down view of the fenestrated endovascular prosthesis of FIG. 7A along plane 7B-7B of FIG. 7A with the secondary endovascular prostheses deployed in fenestration tubes.

FIG. 7C is a bottom-up view of the fenestrated endovascular prosthesis of FIG. 7A along plane 7C-7C of FIG. 7A with the secondary endovascular prostheses deployed in fenestration tubes.

FIG. 8 is a perspective view of a deployment device for the fenestrated endovascular prosthesis of FIGS. 1 and 3.

FIG. 9 is a cross-sectional view of a portion of the deployment device of FIG. 8.

FIG. 10A is a perspective view of a ratchet slide component of the deployment device of FIGS. 8 and 9.

FIG. 10B is a cross-sectional view of the ratchet slide of FIG. 10A.

FIG. 11 is a side view of a carrier of the deployment device of FIGS. 8 and 9.

FIG. 12A is a cross-sectional view of a portion of the deployment device of FIGS. 8 and 9.

FIG. 12B is a partial cut-away view of a portion of the deployment device of FIG. 12A.

FIG. 13 is a cross-sectional view of a portion of the deployment device of FIGS. 8 and 9.

FIG. 14A is a side view of a portion of a delivery catheter assembly of the deployment device of FIGS. 8 and 9.

FIG. 14B is a cross-sectional view of a portion of the delivery catheter assembly of the deployment device of FIGS. 8 and 9 along plane 14B-14B of FIG. 14A.

FIG. 14C is a cross-sectional view of a portion of the delivery catheter assembly of the deployment device of FIGS. 8 and 9 along plane 14C-14C of FIG. 14A.

FIG. 14D is a cross-sectional view of a portion of the delivery catheter assembly of the deployment device of FIGS. 8 and 9 along plane 14D-14D of FIG. 14A.

FIG. 14E is a side view of a portion of the delivery catheter assembly of the deployment device of FIGS. 8 and 9.

FIG. 15A is a side view of an orientation indicium on a distal tip of the deployment device of FIGS. 8 and 9.

FIG. 15B is a side view of an orientation indicium on a distal tip of the deployment device of FIGS. 8 and 9.

FIG. 16A is a side view of a fenestration dilator.

FIG. 16B is a side view of a fenestration dilator.

FIG. 17A is a side view of a fenestration balloon dilator.

FIG. 17B is a side view of a fenestration balloon dilator.

FIG. 18A is a side view of an end portion of a guidewire including a kinked guidewire identifier.

FIG. 18B is a side view of an end portion of a guidewire including a striped guidewire identifier.

FIG. 18C is a side view of an end portion of a guidewire including a ball-type guidewire identifier.

FIG. 19A is a front section view of guidewires of a deployment device inserted into a patient's body, through a treatment site, and out of the patient's body.

FIG. 19B is a side section view of a fenestrated endovascular prosthesis being deployed in a diseased blood vessel.

FIG. 19C is a side section view of the fenestrated endovascular prosthesis of FIG. 19B deployed in the diseased blood vessel.

FIG. 19D is a side section view of fenestration dilators un-sealing fenestration tubes of the fenestrated endovascular prosthesis of FIG. 19C.

FIG. 19E is a side section view of secondary endovascular prostheses deployed in the fenestration tubes of the fenestrated endovascular prosthesis of FIG. 19D.

### DETAILED DESCRIPTION

Degenerative diseases of the arteries of a human body, such as aneurysms and dissections, may be treated by arterial replacement. Conventional open surgery for arterial replacement may be associated with significant risk of death or disability and may be especially dangerous for the vascular patient who typically has significant pre-existing surgical risk factors.

Minimally invasive alternatives to open vascular surgery have been developed, including processes whereby an arterial bypass is performed by placing a tubular endovascular prosthesis within a diseased arterial segment via a remote access point. Such endovascular prostheses may be composed of an impervious fabric wall defining a bore through which blood flows. The impervious fabric prevents blood leakage though the wall of the endovascular prosthesis. The endovascular prosthesis directs blood flow through the bore of the endovascular prosthesis, bypassing the diseased segment of artery. The impervious fabric may be sealed to disease-free arterial walls above and below the diseased segment of artery to be bypassed. Such endovascular prostheses may be utilized to supplant diseased segments of the aorta, such as thoracic and abdominal portions of the aorta, as well as peripheral arteries. Tubular endovascular prostheses may be limited in their ability to maintain flow to branched arteries, as a sealed tubular construct is positioned across openings of respective branched arteries and would prevent blood flow to the branched arteries. Examples of regions of the aorta that may be affected by arterial disease and that include branched arteries include the aortic arch (from which the innominate, carotid, and subclavian arteries originate) and the proximal abdominal aorta (from which the visceral and renal arteries originate).

Various examples relate to an improved endovascular prosthesis, methods of forming the improved endovascular prosthesis, methods of treating a patient using the improved endovascular prosthesis, guidewire identification systems for use with the improved endovascular prosthesis, and a deployment device for deploying the improved endovascular prosthesis in a patient. In some examples, the improved endovascular prosthesis is a fenestrated endovascular prosthesis. The fenestrated endovascular prosthesis can be used to bypass a damaged section of the aorta or other artery, such as a section of the aorta having branch arteries extending from the aorta.

The fenestrated endovascular prosthesis can include a tubular body having a bore, proximal and distal portions configured to couple with healthy arterial tissue, and one or more fenestration tubes, openings, or pockets extending between the proximal and distal portions. Each of the fenestration tubes can include an opening at a proximal end that is in fluid communication with the bore and an opening at a distal end that is in fluid communication with a portion of the artery outside of the fenestrated endovascular prosthesis. The fenestration tubes may extend between a first material and a second material of the fenestrated endovascular prosthesis, such that no holes or openings are formed in the fenestrated endovascular prosthesis. This improves sealing in the fenestrated endovascular prosthesis. Although the fenestration tubes are described as tubes, the fenestration tubes may include openings or pockets formed in layers of the tubular body, and may be defined by the layers of the tubular body or separate materials. The fenestrated endovascular prosthesis can be deployed with the fenestration tubes in a sealed configuration such that blood flow through the fenestration tubes to the exterior of the fenestrated endovascular prosthesis is prevented.

Guidewires may be placed in each of the fenestration tubes and may be used to aid in deploying secondary endovascular prostheses in the fenestration tubes. The secondary endovascular prostheses may be deployed in the fenestration tubes to provide pathways from the bore of the fenestrated endovascular prosthesis to branch arteries adjacent the tubular body of the fenestrated endovascular prosthesis. Providing the fenestrated endovascular prosthesis with the fenestration tubes including guidewires placed therein allows for the fenestrated endovascular prosthesis to be used to repair arteries, even in portions of a patient's body with branched arteries extending from a main artery. The fenestrated endovascular prosthesis can be deployed with the guidewires in place in the fenestration tubes, which aids a practitioner in locating the fenestration tubes and deploying the secondary endovascular prostheses therein.

A method of manufacturing a fenestrated endovascular prosthesis may include obtaining a body-forming mandrel for forming the body of the fenestrated endovascular prosthesis. The body-forming mandrel may be partially covered with a first material structure, such as with one or more layers of a biocompatible material. Fenestration tubes or fenestration tube mandrels may be positioned on the first material structure in a desired pattern with proximal ends of the fenestration tubes/fenestration tube mandrels being disposed proximally to proximal edges of the first material structure. The body-forming mandrel, the first material structure, and the fenestration tubes/fenestration tube mandrels may be partially covered with a second material structure with distal edges of the second material structure being disposed proximally to distal ends of the fenestration tubes/fenestration tube mandrels. In examples in which the fenestration tube mandrels are placed between the first material structure and the second material structure, the fenestration tube mandrels can be removed after sealing the second material structure to the first material structure, thus forming fenestration tubes as openings, pockets, or passageways between the first material structure and the second material structure. The fenestration tubes can include lumens through which blood may eventually flow, and the lumens may be in a sealed configuration. A wire stent may be coupled to the first and second material structures. Guidewires may be placed in each of the fenestration tubes prior to the fenestrated endovascular prosthesis being crimped into a delivery configuration. This method of manufacturing the fenestrated endovascular prosthesis allows for a simple cylindrical-shaped body-forming mandrel to be used, allows for the fenestration tubes to be positioned in any desired configuration, and provides for good sealing between the various components of the fenestrated endovascular prosthesis (e.g., the first material structure, the second material structure, and the fenestration tubes).

A method of repairing a diseased portion of an artery (e.g., a portion of the aorta) or a blood vessel having one or more branch arteries may include providing a fenestrated endovascular prosthesis, which includes fenestration tubes and guidewires extending through the fenestration tubes. The guidewires may be advanced into an insertion site (e.g., one of a jugular, femoral, or other access site), through a treatment site, and out of an extraction site (e.g., the other of a jugular, femoral, or other access site). A deployment device can be used to advance the fenestrated endovascular prosthesis to the treatment site. The deployment device may be used to deploy the fenestrated endovascular prosthesis at the treatment site, and may then be removed. The deployment device can slide along the guidewires as the deployment device is removed. The guidewires can be used to open selected fenestration tubes of the fenestrated endovascular prosthesis, and can be used to deploy secondary endovascular prostheses between the selected fenestration tubes and branch arteries extending from the treatment site. The guidewires can be removed. Fenestration tubes that are not selected for the deployment of the secondary endovascular prostheses can be left in a sealed configuration. This method of repairing an artery or blood vessel provides a practitioner with easy access to fenestration tubes in the fenestrated endovascular prosthesis and aids in forming blood flow pathways between the fenestrated endovascular prosthesis and branch arteries adjacent the treatment site.

Guidewire identification features can be provided on the guidewires in order to identify specific guidewires that extend through each of the fenestration tubes of the fenestrated endovascular prosthesis, even when the fenestrated endovascular prosthesis is positioned within a patient and is not directly visible. The guidewire identification features may include kinks, marker bands, marker nubs, or the like, which are provided on each of the guidewires. A different number of guidewire identification features may be included on each of the guidewires in order to identify each of the guidewires. Each of the guidewires may include the same guidewire identification features at each end of the respective guidewire. Providing the guidewire identification features allows a practitioner to identify which guidewire extends through which fenestration tube in a deployed fenestrated endovascular prosthesis, and deploy secondary endovascular prostheses or the like along selected guidewires to selected fenestration tubes.

A deployment device for the fenestrated endovascular prosthesis may include various channels, grooves, and the like for receiving guidewires that extend through fenestration tubes of the fenestrated endovascular prosthesis. For example, grooves may be formed in a distal tip of the deployment device. The guidewires may extend in the grooves and into an outer sheath of the deployment device. The guidewires may extend into a bore of the fenestrated endovascular prosthesis, through the fenestration tubes, and along an exterior of the fenestrated endovascular prosthesis. Guide lumens may be provided in an intermediate sheath of the deployment device, and the guidewires may extend through the guide lumens and into a handle assembly of the deployment device. The guidewires may then extend out of the handle assembly near a luer fitting of the deployment device. Providing grooves, channels, and the like in the deployment device for slidably receiving the guidewires allows the guidewires to be positioned in a patient and in the fenestrated endovascular prosthesis before the deployment device and the fenestrated endovascular prosthesis are advanced to a treatment site within the patient's body. The deployment device may then be advanced to the treatment site, the fenestrated endovascular prosthesis may be deployed, and the deployment device may retreat along the guidewires and out of the patient's body.

Embodiments may be understood by reference to the drawings, wherein like parts are designated by like numerals throughout. It will be understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of an implanted medical device means the end of the device furthest from the heart. The proximal end refers to the opposite end, or the end nearest the heart. As specifically applied to a fenestrated endovascular prosthesis, the proximal end of the prosthesis refers to the end configured for deployment nearest the heart (along the blood flow path of the vasculature) and the distal end refers to the opposite end, the end farthest from the heart. If at one or more points in a procedure a physician changes the orientation of the prosthesis, as used herein, the term “proximal end” always refers to the end configured for deployment closest to the heart when implanted. Additionally, the proximal end of a deployment device means the end of the deployment device proximal a handle assembly. The distal end refers to the opposite end of the deployment device where the distal tip is located. The distal tip at the distal end of the delivery device may be inserted at an insertion site in a patient, and the proximal end and the handle assembly of the delivery device may remain outside the patient.

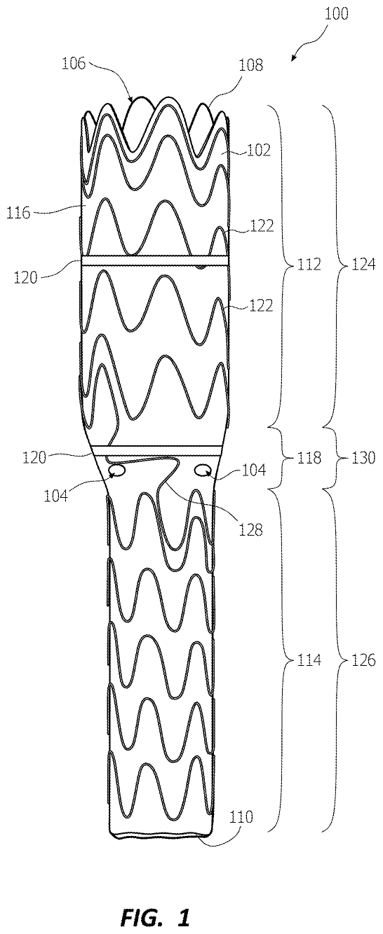

FIGS. 1 through 19D illustrate various views of a fenestrated endovascular prosthesis and related components. In certain views a fenestrated endovascular prosthesis may be coupled to, or shown with, additional components that are not included in every view. Further, in some views, only selected components are illustrated in order to provide details of the relationships between the components. Some components may be shown in multiple views, but not discussed in connection with every view. Disclosure provided in connection with any figure is relevant and applicable to the disclosure provided in connection with any other figure, example, or embodiment.

FIG. 1 illustrates a fenestrated endovascular prosthesis 100, in accordance with some examples. The fenestrated endovascular prosthesis 100 can be placed within a diseased arterial segment in order to supplant the diseased segment of artery. The fenestrated endovascular prosthesis 100 can include a tubular body 102 and one or more fenestration tubes 104 extending between a bore 106 of the tubular body 102 and an exterior of the tubular body 102. The fenestration tubes 104 can include tubes, openings, pockets, pathways, or the like, and can be referred to generally as fenestration tubes or fenestrations. In some examples, the fenestration tubes 104 can be included to provide pathways between the bore 106 of the tubular body 102 and branched arteries or the like. As will be discussed in detail below, secondary endovascular prostheses may be deployed in the fenestration tubes 104 in order to direct blood flow from the bore 106 of the tubular body 102, through the fenestration tubes 104, to the outside of the tubular body 102, such as to the branched arteries.

The tubular body 102 may be generally cylindrical in shape. The tubular body 102 may include a proximal end 108 and a distal end 110. The tubular body 102 may include a proximal portion 112 extending distally from the proximal end 108, a distal portion 114 extending proximally from the distal end 110, and the bore 106 (e.g., an interior of the tubular body 102). The bore 106 may be defined by a wall 116 extending from the proximal end 108 through the proximal portion 112 and the distal portion 114 to the distal end 110. The tubular body 102 may be formed of a variety of materials and/or layers of materials. Suitable materials for the tubular body 102 include biocompatible materials that are resistant to the passage of blood and/or cells through the wall 116 of the tubular body 102. In some examples, the tubular body 102 may be formed of polyethylene terephthalate, polyurethane, silicone rubber, nylon, fluoropolymer, polyester, combinations or multiple layers thereof, or the like. A thickness of the wall 116 of the tubular body 102 may be in a range from about 0.07 mm to about 0.5 mm. In some examples, a length of the tubular body 102 can range from about 50 mm to about 250 mm. An outer diameter of the tubular body 102 can range from about 18 mm to about 55 mm. In some examples, a tapered portion 118 can be disposed between the proximal portion 112 and the distal portion 114. The tapered portion 118 can be used to provide the distal portion 114 with a smaller diameter than the proximal portion 112. The tapered portion 118 can taper at an angle in a range from about 5° to about 90°, from about 10° to about 60°, or the like. In some examples, the tubular body 102 can taper radially inward from the proximal portion 112 to the distal portion 114 over the length of the tubular body 102, such as from the proximal end 108 to the distal end 110. In examples in which the tubular body 102 is tapered radially inward form the proximal end 108 to the distal end 110, the taper angle can be in a range from about 2° to about 15°. In some examples, the taper angle may be a position along the length of the tapered portion 118 between a first diameter at the distal end 110 and a second diameter at the proximal end 108 of the tubular body 102. Two fenestration tubes 104 are illustrated in the example of FIG. 1. However, the tubular body 102 may have any number of the fenestration tubes 104, such as four fenestration tubes 104, or more or fewer fenestration tubes 104. The wall 116 of the tubular body 102 may include an inner material structure and an outer material structure circumferentially surrounding at least a portion of the inner material structure and the fenestration tubes 104. The inner material structure can be provided in the distal portion 114 and the tapered portion 118. The outer material structure can be provided in the proximal portion 112 and the tapered portion 118. In some examples, the inner material structure can further extend into the proximal portion 112 and/or the outer material structure can further extend into the distal portion 114. The fenestration tubes 104 may be included in the fenestrated endovascular prosthesis 100 to provide a pathway from the bore 106 of the tubular body 102, through the wall 116, to the outside of the tubular body 102, along the length of the tubular body 102. In other words, the fenestration tubes 104 may provide a pathway for blood to travel from inside the bore 106 to an exterior of the fenestrated endovascular prosthesis 100, such as to branched arteries or the like. In some examples, the fenestration tubes 104 can be openings, pockets, or pathways, and may be formed from materials of the tubular body 102, such as the inner material structure and/or the outer material structure.

Each of the fenestration tubes 104 may extend between the inner material structure and the outer material structure of the wall 116 of the tubular body 102. In some examples, the fenestration tubes 104 may extend from proximal or level with a proximal end or proximal portion of the inner material structure to distal or level with a distal end or distal portion of the outer material structure. As such, the inner material structure and the outer material structure may not include openings through which the fenestration tubes 104 extend, and the fenestration tubes 104 may not be blocked by the inner material structure and the outer material structure. This increases the strength and durability of the fenestrated endovascular prosthesis 100. In some examples, slits or openings may be formed in the inner material structure and/or the outer material structure, and the fenestration tubes 104 may extend through the slits or openings. In some examples, the fenestration tubes 104 may be openings, pockets, or pathways formed between the inner material structure and the outer material structure, such that the fenestration tubes 104 extend from proximal to distal from a proximal end or proximal portion of the inner material structure to a distal end or distal portion of the outer material structure.

The tubular body 102 may include radiopaque marker bands 120 or other indicia at, adjacent to, or near proximal ends and/or distal ends of the fenestration tubes 104. In some examples, the tubular body 102 may include radiopaque marker bands or other indicia at, adjacent to, or near proximal ends and/or distal ends of the tubular body 102 in addition to, or in alternative to the radiopaque marker bands 120. The radiopaque marker bands 120 can be formed from any suitable radiopaque material, such as gold, tantalum, or platinum-iridium alloy. Other materials are contemplated within the scope of this disclosure.

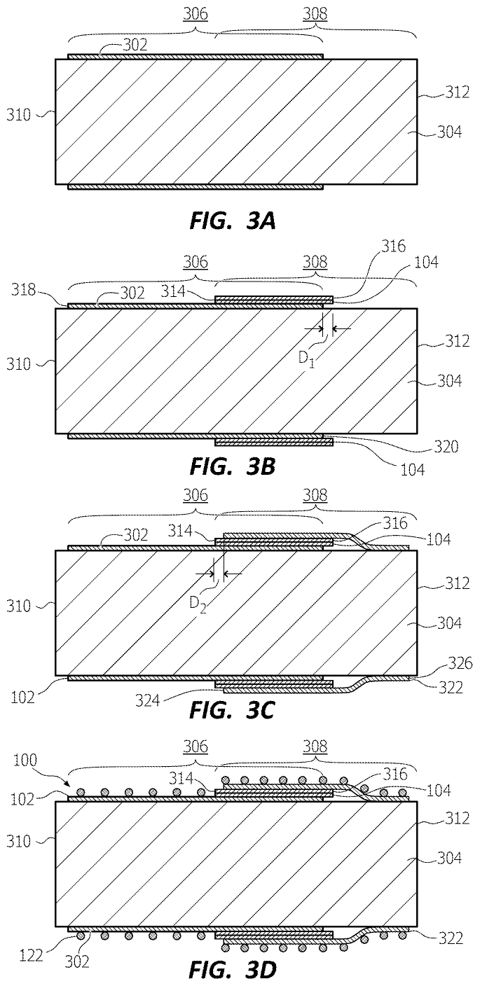

In the example illustrated in FIG. 1, each of the fenestration tubes 104 can extend from proximal or even with a proximal end or proximal portion of the inner material structure of the tubular body 102 at a position of a top-most one of the radiopaque marker bands 120 to distal or even with a distal end or distal portion of the outer material structure of the tubular body 102 at a position of the lower-most one of the radiopaque marker bands 120. Additional details of the inner material structure and the outer material structure of the tubular body 102 are illustrated and discussed below with reference to FIGS. 3A through 3D. The fenestration tubes 104 provide access between the bore 106 of the fenestrated endovascular prosthesis 100 and the exterior of the tubular body 102, and may be used as access points for additional prostheses (e.g., secondary endovascular prostheses or the like), discussed in detail below. The radiopaque marker bands 120 may be used in conjunction with radiography, fluoroscopy, or the like to aid in the deployment of the additional prostheses or the like within the fenestration tubes 104.

A wire stent 122 may be attached to the tubular body 102 and may circumferentially surround the tubular body 102. In some examples, the wire stent 122 can be disposed inside the tubular body 102 and can be circumferentially surrounded by the tubular body 102, or can be disposed between adjacent layers of the wall 116 of the tubular body 102. The wire stent 122 may be a wire scaffold, a framework, a stent, or the like. The wire stent 122 may be configured to provide a desired shape to the fenestrated endovascular prosthesis 100. The wire stent 122 may be configured to radially expand the tubular body 102 from a crimped or delivery configuration to a deployed configuration. The wire stent 122 can be formed of an elastic material (e.g., a super-elastic material), which allows for the wire stent 122 to self-expand from the delivery configuration to the deployed configuration. In some examples, the wire stent 122 can be formed of nickel-titanium alloy, stainless steel, platinum, polymers, combinations or multiple layers thereof, or the like.

The fenestrated endovascular prosthesis 100 can be provided in a deployment device in the delivery configuration. Once the fenestrated endovascular prosthesis 100 is placed in a desired location in a patient's body, the fenestrated endovascular prosthesis 100 can be deployed and the wire stent 122 can cause the tubular body 102 to expand to the deployed configuration. When the fenestrated endovascular prosthesis 100 is deployed within a blood vessel, the tubular body 102 can be pressed against a wall of the blood vessel by a spring force of the wire stent 122. This seals the fenestrated endovascular prosthesis 100 to the wall of the blood vessel, which forces blood to flow through the tubular body 102. As such, the fenestrated endovascular prosthesis 100 is used to bypass a damaged section of the blood vessel. Additionally, or alternatively, stents configured for balloon expansion or self-expansion driven by material properties of the tubular body 102 or other elements are likewise within the scope of this disclosure.

The wire stent 122 may have a zig-zag pattern, a wave pattern, or any other suitable pattern. The wire stent 122 can include a proximal stent portion 124 that overlays the proximal portion 112 of the tubular body 102 and a distal stent portion 126 that overlays the distal portion 114 of the tubular body 102. The wire stent 122 can further include a spine 128 extending between the proximal stent portion 124 and the distal stent portion 126. The spine 128 can provide a generally stent-free portion 130 of the tubular body 102 between the proximal portion 112 and the distal portion 114. As illustrated in FIG. 1, the stent-free portion 130 can have a tapered shape. The wire stent 122 can be pre-formed, or formed over the tubular body 102. The material, pattern, and wire diameter of the wire stent 122 may be configured to provide a continuous force directed radially outward, and a continuous resistance to forces directed radially inward. Additionally, embodiments wherein a tapered portion between the proximal portion 112 and distal portion 114 includes wire patterns or other stent structures are also within the scope of this disclosure.

In some examples, the proximal portion 112 of the tubular body 102 and/or the distal portion 114 of the tubular body 102 may include flared ends. The flared ends can facilitate scaling of the tubular body 102 (e.g., sealing of the proximal portion 112 and/or the distal portion 114) with a wall of a blood vessel, and can prevent leakage of blood between the tubular body 102 and portions of the blood vessel to be treated. The flared ends may be achieved by providing the wire stent 122 with a greater diameter at the proximal end 108 of the proximal portion 112 and/or the distal end 110 of the distal portion 114.

In some examples, the tubular body 102 may include a cuff, which can be disposed adjacent to the proximal end 108 and/or the distal end 110. The cuff can be configured to facilitate sealing of the proximal portion 112 and/or the distal portion 114 of the fenestrated endovascular prosthesis 100 with a wall of a blood vessel, and can prevent leakage of blood between the tubular body 102 and portions of the blood vessel to be bypassed. In some examples, the cuff can be formed of a relatively porous material, as compared to the wall 116 of the tubular body 102. This may allow for or facilitate tissue ingrowth into the cuff, thus sealing the cuff of the tubular body 102 to the patient's body.

In some examples, fixation features may be formed in the tubular body 102, such as along the outside of the tubular body 102. The fixation features can be configured to prevent migration of the fenestrated endovascular prosthesis 100 relative to a wall of a blood vessel once the fenestrated endovascular prosthesis 100 is placed and deployed inside of a patient's body. The fixation features can include protruding barbs, sharpened protruding barbs, an adhesive, inflatable portions, combinations or multiples thereof, or the like.

In some examples, the wall 116 of the tubular body 102 may be formed of materials that are impermeable to tissue cell ingrowth into and/or tissue cell migration through the wall 116. This may prevent or discourage stenosis of the tubular body 102. The wall 116 can be impermeable to blood such that blood is prevented from leaking from the bore 106 of the fenestrated endovascular prosthesis 100 to the exterior of the fenestrated endovascular prosthesis 100 and into surrounding tissue. In some examples, interior surfaces of the wall 116 may include serially deposited fibers of polytetrafluoroethylene (PTFE), which can resist fibrin deposition and platelet adhesion on the interior surfaces of the wall 116.

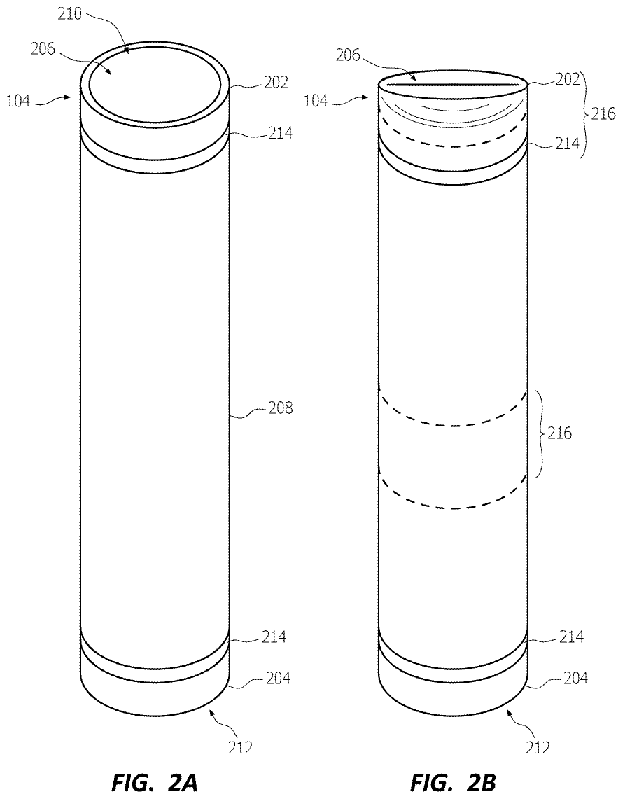

FIGS. 2A and 2B illustrate a fenestration tube 104 in an open configuration and a sealed configuration, respectively. As discussed in reference to FIG. 1, fenestration tubes 104 may be included in a fenestrated endovascular prosthesis 100 to provide access between a bore 106 of a tubular body 102 and an exterior of the fenestrated endovascular prosthesis 100. For example, the fenestration tubes 104 may provide access between the bore 106 of the tubular body 102 and a branched arteries outside the fenestrated endovascular prosthesis 100, and secondary endovascular prostheses may be deployed in any of the fenestration tubes 104 to provide pathways between the bore 106 and the branched arteries.

In the illustrated embodiment, the fenestration tube 104 is shown as a distinct tube, separate from the endovascular prosthesis 100 or the tubular body 102. However, as discussed below, embodiments wherein the fenestration tube 104 is formed from other elements of the endovascular prosthesis 100 or tubular body 102 are within the scope of this disclosure. For example, the fenestration tube 104 may comprise a pocket or opening disposed between inner and outer layers of the tubular body 102. Thus, the fenestration tube 104 may include walls formed and defined by portions of the tubular body 102. The fenestration tube 104 may not have walls or a body separate from the tubular body 102. Whether or not the fenestration tube 104 comprises its own walls, or the walls of the fenestration tube 104 are portions of layers of the tubular body 102, FIGS. 2A and 2B are helpful in identifying and describing portions of the fenestration tube 104. Accordingly, the description of FIGS. 2A and 2B applies to fenestration tubes 104 with discrete walls or tubes separate from the tubular body 102 and fenestration tubes 104 formed by creating an opening or pocket between layers or elements of the tubular body 102. As an example, a fenestration tube 104 may be formed by placing a mandrel along a portion of the tubular body 102 during manufacture, covering the mandrel with elements that create the tubular body 102, and removing the mandrel to leave a pocket that forms the fenestration tube 104.

In FIG. 2A, the fenestration tube 104 may include a proximal end 202, a distal end 204, and a lumen 206 defined by a fenestration tube wall 208 extending from the proximal end 202 to the distal end 204. The fenestration tube wall 208 may be formed of a variety of materials and/or layers of materials. Suitable materials for the fenestration tube wall 208 include biocompatible materials that are resistant to the passage of blood and/or cells through the fenestration tube wall 208 of the fenestration tube 104. The fenestration tube 104 may be formed from any of the materials listed for forming the tubular body 102, described above with respect to FIG. 1. In some examples, the fenestration tube 104 may be formed of the same material as the tubular body 102. In some examples, the fenestration tube 104 and the tubular body 102 can be formed of different materials. Again, as noted above, the fenestration tube 104 may be formed by an opening or pocket in the tubular body 102. Thus, the fenestration tube wall 208 may be formed by portions or layers of the tubular body 102 and may not be separate from the tubular body 102.

The lumen 206 may include a proximal opening 210 disposed at the proximal end 202 that provides access for fluid communication into the lumen 206. The proximal opening 210 may be in fluid communication with the bore 106 of the fenestrated endovascular prosthesis 100. The lumen 206 may include a distal opening 212 disposed at the distal end 204 that provides access for fluid communication out of the lumen 206. The distal opening 212 may be in fluid communication with an exterior of the fenestrated endovascular prosthesis 100.

The lumen 206 of the fenestration tube 104 may be configured to receive a secondary endovascular prosthesis. For example, as will be discussed in detail below, a guidewire may be placed in the fenestration tube 104. The guidewire may be used to provide access to the fenestration tube 104, such as to un-seal the fenestration tube from the sealed configuration to the open configuration, to place a secondary endovascular prosthesis in the fenestration tube 104, and the like. The secondary endovascular prosthesis may be deployed in the fenestration tube 104 and may provide a path for blood to flow between the bore 106 of the fenestrated endovascular prosthesis 100 and an exterior of the fenestrated endovascular prosthesis 100, such as to a branched artery.

Radiopaque marker bands 214 or other indicia can be disposed adjacent or near the proximal end 202 and/or the distal end 204 of the fenestration tube 104. The radiopaque marker bands 214 can be formed from any suitable radiopaque material, such as gold, tantalum, or platinum-iridium alloy. Other materials are contemplated within the scope of this disclosure. The radiopaque marker bands 214 may be used in conjunction with radiography, fluoroscopy, or the like to aid in the positioning and aligning of a secondary endovascular prosthesis relative to the fenestration tube 104. In FIGS. 2A and 2B the radiopaque marker bands 214 are shown disposed around the fenestration tube 104. Embodiments wherein the radiopaque marker bands are disposed around the tubular body 102 at longitudinal positions corresponding to the proximal end 202 and/or distal end 204 of the fenestration tube 104 are likewise within the scope of this disclosure.

Regardless of whether the fenestration tube wall 208 is formed from portions of the tubular body 102 or whether the fenestration tube wall 208 is a separately formed component, in some examples, the fenestration tube wall 208 of the fenestration tube 104 may be formed of materials that are impermeable to tissue cell ingrowth into and/or tissue cell migration through the fenestration tube wall 208. This may prevent or discourage stenosis of the fenestration tube 104. The fenestration tube wall 208 can be impermeable to blood such that blood is prevented from leaking from inside a fenestrated endovascular prosthesis 100 including the fenestration tube 104 to the exterior of the fenestrated endovascular prosthesis 100 and into surrounding tissue. In some examples, interior surfaces of the fenestration tube wall 208 may include serially deposited fibers of PTFE, which can resist fibrin deposition and platelet adhesion on the interior surfaces of the fenestration tube wall 208.

The fenestration tube 104 can include a straight cylindrical shape, as illustrated in FIG. 2A. In some examples, the fenestration tube 104 may include any suitable transverse cross-sectional shape, such as oval, obround, semicircular, D-shaped, or the like. A length of the fenestration tube 104 may range from about 5 mm to about 50 mm. A thickness of the fenestration tube wall 208 may range from about 0.07 mm to about 0.5 mm. A diameter of the lumen 206 may range from about 2 mm to about 30 mm.

As illustrated in FIG. 2B, the fenestration tube 104 may include a seal region 216, which prevents blood flow through the lumen 206 (e.g., from a bore 106 of a fenestrated endovascular prosthesis 100 to an exterior of the fenestrated endovascular prosthesis 100). The seal region 216 may be present when the fenestration tube 104 is in the sealed configuration, and the seal region 216 may be opened or un-sealed when the fenestration tube 104 is in the open configuration. The seal region 216 can be configured to be selectively opened when the fenestration tube 104 is transitioned from the sealed configuration to the open configuration, as will be described in detail below. In some examples, the seal region 216 may be configured to be partially opened or unsealed. For example, the seal region 216 may be partially opened when a respective fenestration tube 104 accepts a guidewire and the seal region 216. A fenestration dilator, examples of which are discussed in detail below, can be used to completely open the fenestration tube 104 (e.g., transfer the fenestration tube 104 to the open configuration) such that the fenestration tube 104 can accept a secondary endovascular prosthesis.

FIG. 2B illustrates two potential locations for the seal region 216. In addition, in some examples, the seal region 216 may be disposed adjacent to the proximal end 202 of the fenestration tube 104. In some examples, the seal region 216 may be disposed centrally along a length of the fenestration tube 104. In some examples, the seal region 216 may be disposed adjacent to the distal end 204 of the fenestration tube 104. A length of the seal region 216 can range from about 2 mm to about 8 mm, such as about 5 mm. The seal region 216 can include an adhesive material, such as fluorinated ethylene propylene (FEP), polyurethane, silicone, the like, any other suitable adhesive, or the like. In some examples, the seal region 216 can be formed by dispensing an adhesive material at the seal region 216 and positioning non-stick material strips, such as polyimide films, within the lumen 206 proximal and distal the seal region 216. The fenestration tube 104 can be radially compressed at the seal region 216 to selectively seal or close the lumen 206 at the seal region 216, and the non-stick material strips can be removed. In some examples, the lumen 206 may be closed using any suitable technique, such as heat-sealing, radio-frequency welding, ultrasound welding, or the like.

FIGS. 3A through 3D are cross-sectional views of a method of manufacturing a fenestrated endovascular prosthesis 100 (illustrated in FIG. 3D). The fenestrated endovascular prosthesis 100 of FIGS. 3A through 3D can be substantially similar to, including some or all of the features of, the fenestrated endovascular prosthesis 100 described with respect to FIG. 1. In FIG. 3A, a first material structure 302 is formed on a body-forming mandrel 304. The body-forming mandrel 304 may include a distal portion 306 extending from a distal end 310 and a proximal portion 308 extending from a proximal end 312. In FIGS. 3A through 3D, the left end of the body-forming mandrel 304 is the distal end 310, and the right end of the body-forming mandrel 304 is the proximal end 312.

The body-forming mandrel 304 may be generally cylindrical in shape and formed from any suitable material, such as stainless steel, aluminum, other metals or metal alloys, plastics, polymers, or the like. In some examples, the body-forming mandrel 304 may include any suitable transverse cross-sectional shape, such as oval, obround, semicircular, D-shaped, or the like. Additionally, the body-forming mandrel 304 may transition from any suitable transverse cross-sectional shape to any other suitable cross-sectional shape along the length of the body-forming mandrel 304. The dimensions (e.g., the length and the diameter) of the body-forming mandrel 304 may correspond to the dimensions of the tubular body 102, as described previously with respect to FIG. 1.

In FIG. 3A, the distal portion 306 of the body-forming mandrel 304 is covered with the first material structure 302. The first material structure 302 may include any of the materials of the tubular body 102, as described previously with respect to FIG. 1. The first material structure 302 may be applied using any suitable technique. For example, the first material structure 302 may be applied by wrapping strips of material around the body-forming mandrel 304, dipping the body-forming mandrel 304 into a material solution, spraying the body-forming mandrel 304 with the material solution, or the like. In some examples, the first material structure 302 may include multiple layers of the same material or different materials. The first material structure 302 may be referred to as a first material layer, and may include multiple sub-layers of the same material or different materials.

In FIG. 3B, two fenestration tubes 104 are positioned on the first material structure 302. The fenestration tubes 104 may be positioned on the first material structure 302 such that a proximal end 316 of each fenestration tube 104 is proximal to a proximal edge 320 of the first material structure 302. The proximal end 316 of each fenestration tube 104 can be disposed proximal to the proximal edge 320 of the first material structure 302 by a distance Di in a range from about 0 mm to about 10 mm, such as about 2 mm. The proximal end of 316 of each fenestration tube 104 can be in contact with the body-forming mandrel 304, which can ensure that the respective fenestration tube 104 is in communication (e.g., fluid communication) with the interior of the tubular body 102. A distal end 314 of each fenestration tube 104 can be between a distal edge 318 of the first material structure 302 and the proximal edge 320 of the first material structure 302.

One, two, three, four or more fenestration tubes 104 may be positioned on the first material structure 302 in any suitable pattern. For example, four fenestration tubes 104 may be positioned on the first material structure 302 with about 90° separating each fenestration tube 104 from adjacent fenestration tubes 104. In some examples, four fenestration tubes 104 may be positioned on the first material structure 302 with greater than 90° separating one fenestration tube 104 from an adjacent fenestration tube 104 and less than 90° separating the fenestration tube from another adjacent fenestration tube 104. Moreover, although the fenestration tubes 104 are illustrated as being positioned at the same point along the length of the first material structure 302 and the body-forming mandrel 304, each of the fenestration tubes 104 may be positioned at any point along the length of the first material structure 302 and the body-forming mandrel 304. For example, a first fenestration tube 104 may be positioned proximally relative to a second fenestration tube 104. In some examples, the fenestration tubes 104 may have different lengths and/or different diameters. The fenestration tubes 104 may be positioned on the first material structure 302 in a patient-specific configuration (e.g., dependent upon relative positions of branched arteries and a damaged section of a blood vessel in a particular patient), in a deployment-specific configuration (e.g., dependent upon where in a patient's body the fenestrated endovascular prosthesis 100 is to be positioned and deployed), or the like. The fenestration tubes 104 may be adhered to the first material structure 302 using an adhesive, such as FEP, polyurethane, silicone, the like, or any other suitable adhesive.

The fenestration tubes 104 may be positioned on the first material structure 302 in a scaled configuration, an open configuration, or a partially scaled configuration. In some examples, the fenestration tubes 104 may be positioned on the first material structure 302 with guidewires extending through the fenestration tubes 104. In some examples, the fenestration tubes 104 may be positioned on the first material structure 302 in the open configuration and may be sealed to the sealed configuration before additional processing of the fenestrated endovascular prosthesis 100. In some examples, the fenestration tubes 104 may be positioned on the first material structure 302 in the open configuration, guidewires may be advanced through the fenestration tubes 104, and the fenestration tubes 104 may be sealed to the sealed configuration.

In FIG. 3C, a second material structure 322 is formed on the body-forming mandrel 304. The proximal portion 308 of the body-forming mandrel 304, portions of the fenestration tubes 104, and a portion of the first material structure 302 can be covered by the second material structure 322. The distal end 314 of each fenestration tube 104 can be disposed distal to a distal edge 324 of the second material structure 322 by a distance D2 in a range from about 0 mm to about 10 mm, such as about 2 mm. In some examples, the distal end 314 of each fenestration tube 104 can be disposed proximal to a distal edge 324 of the second material structure 322 by a distance in a range of greater than about 0 mm to about 10 mm. This may result in the distal end 314 of the respective fenestration tube 104 being effectively ‘closed.’ A proximal end 316 of each fenestration tube 104 can be between the distal edge 324 of the second material structure 322 and a proximal edge 326 of the second material structure 322. The combination of the first material structure 302, the fenestration tubes 104, and the second material structure 322 forms the tubular body 102 of the fenestrated endovascular prosthesis 100, described above with reference to FIG. 1.

The second material structure 322 may be applied using any suitable technique. For example, the second material structure 322 may be applied by wrapping strips of material around the construct of FIG. 3B, dipping the construct of FIG. 3B into a material solution, spraying the construct of FIG. 3B with the material solution, or the like. In some examples, the second material structure 322 may include multiple layers of the same material or different materials. The second material structure 322 may be referred to as a second material layer, and may include multiple sub-layers of the same material or different materials.

In some examples, the fenestration tubes 104 may be omitted, and fenestration tube mandrels or other non-stick elements can be placed between the first material structure 302 and the second material structure 322 in the same positions as the fenestration tubes 104. In some examples, the fenestration tube mandrels can be formed of materials the same as or similar to the body-forming mandrel 304. After the first material structure 302 is formed on the body-forming mandrel 304 in FIG. 3A, fenestration tube mandrels can be placed in the same positions as the fenestration tubes 104, similar to FIG. 3B. The second material structure 322 can then be formed around the proximal portion 308 of the body-forming mandrel 304, portions of the fenestration tube mandrels, and a portion of the first material structure 302. The second material structure 322 can be sealed to the first material structure 302 around the fenestration tube mandrels. For example, the second material structure 322 and/or the first material structure 302 can be heated, sintered, melted, or otherwise sealed around the fenestration tube mandrels. The fenestration tube mandrels can then be removed to form the fenestration tubes 104 as openings, pockets, passageways, or the like along the tubular body 102, between the first material structure 302 and the second material structure 322. In such examples, the fenestration tubes 104 include materials of the first material structure 302 and the second material structure 322. Any description herein relating to the fenestration tubes 104 applies to fenestration tubes 104 created as pockets, openings, or passageways along the tubular body 102. For example, the fenestration tubes 104 created as pockets, openings, or passageways along the tubular body 102 can be sealed by any of the methods described in connection with sealing fenestration tubes 104, above.

In FIG. 3D, a wire stent 122 is coupled to the first material structure 302 and the second material structure 322. In other words, the wire stent 122 is coupled to the tubular body 102. The wire stent 122 may circumferentially surround the tubular body 102. The combination of the first material structure 302, the fenestration tubes 104, the second material structure 322, and the wire stent 122 forms a fenestrated endovascular prosthesis 100.

The wire stent 122 may be pre-formed on a wire bending mandrel prior to placement over the first material structure 302 and the second material structure 322. In some examples, the wire stent 122 may be wound around the first material structure 302 and the second material structure 322 and may be formed on the first material structure 302, the second material structure 322, and the body-forming mandrel 304 in situ. The wire stent 122 may be coupled to the first material structure 302 and the second material structure 322 using any suitable technique. For example, the wire stent 122 may be coupled to the first material structure 302 and the second material structure 322 using an adhesive, suture stitches, clips, staples, tapes, films, membranes, combinations thereof, or the like.

In some examples, the wire stent 122 may be captured between material layers of the first material structure 302 and/or the second material structure 322. For example, the wire stent 122 may be formed between adjacent layers of the first material structure 302; between adjacent layers of the second material structure 322; between the first material structure 302 and the second material structure 322, or any combination thereof. In some examples, the wire stent 122 can be formed around the first material structure 302 and the second material structure 322, and a third material structure (not separately illustrated), which may be formed of materials and by processes similar to or the same as the first material structure 302 and the second material structure 322, can be formed around the wire stent 122. In some examples, the wire stent 122 can initially be coupled to the body-forming mandrel 304 and the first material structure 302, the fenestration tubes 104, and the second material structure 322 can be applied around the wire stent 122.

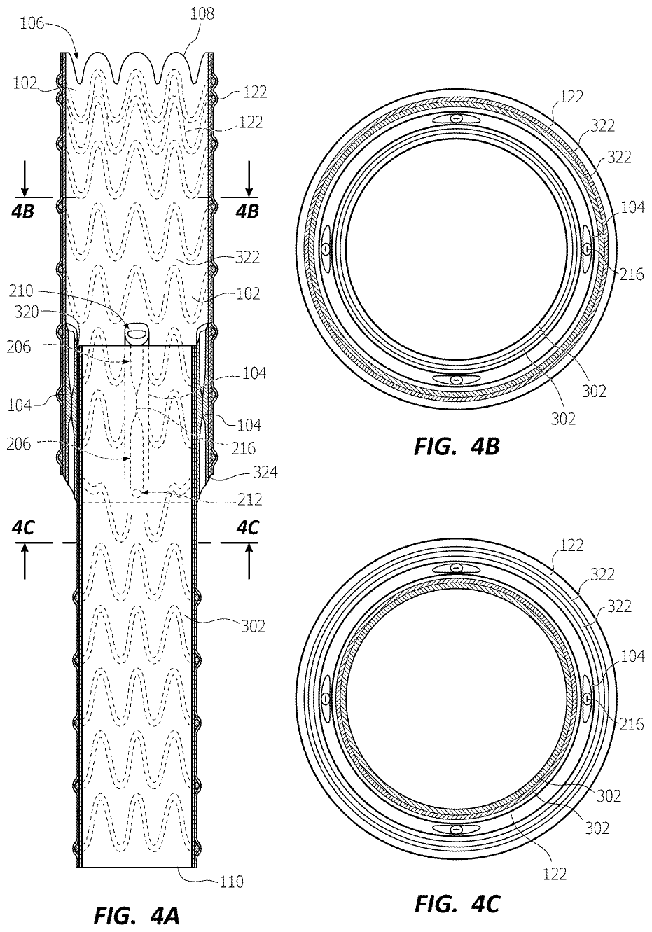

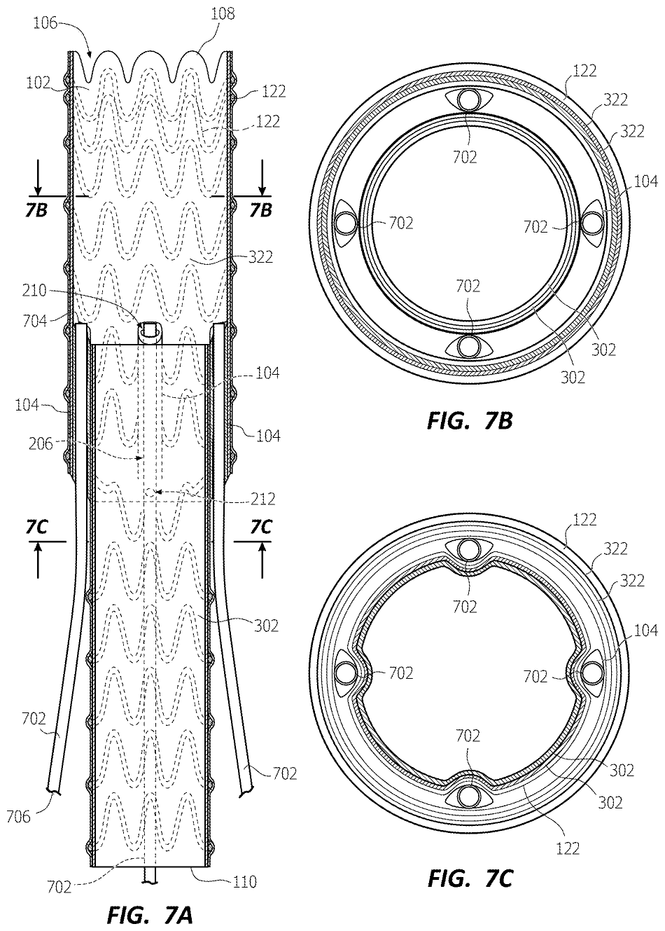

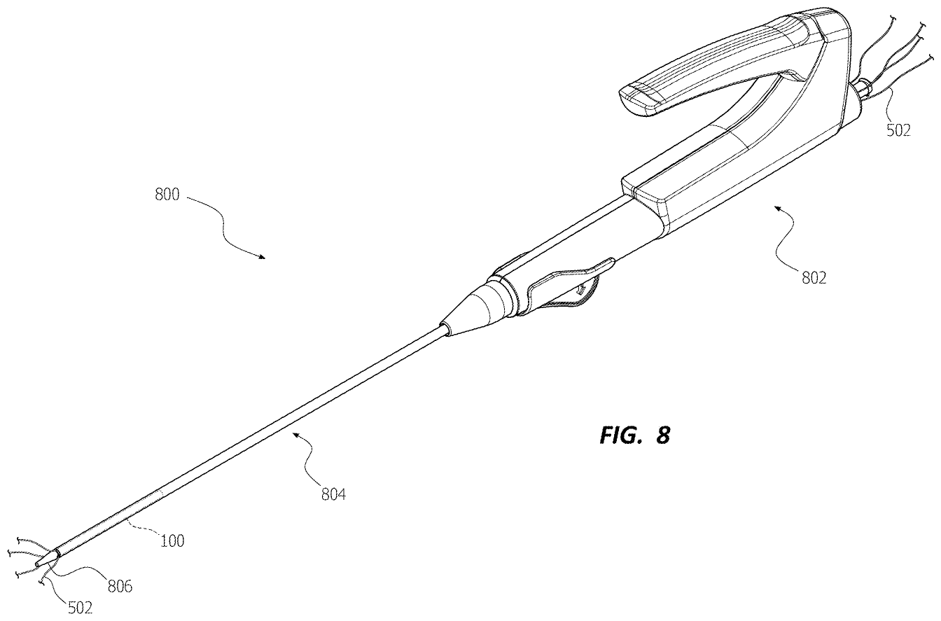

FIGS. 4A through 7C illustrate a method of deploying a secondary endovascular prosthesis 700 (illustrated in FIGS. 7A through 7C) in a fenestrated endovascular prosthesis 100. The fenestrated endovascular prosthesis 100 may be the same as or similar to the fenestrated endovascular prosthesis 100 discussed above with respect to FIGS. 1 and 3A through 3D. FIGS. 4A through 4C illustrate a side section view, a top-down view, and a bottom-up view of the fenestrated endovascular prosthesis 100. The fenestrated endovascular prosthesis 100 can include fenestration tubes 104 in a sealed configuration. For example, a lumen 206 of each of the fenestration tubes 104 can include a seal region 216, which can be provided proximally, centrally, or distally along a length of the respective fenestration tube 104.

The fenestrated endovascular prosthesis 100 includes a proximal portion 112 extending from a proximal end 108. The proximal portion 112 can be defined by a second material structure 322. The fenestrated endovascular prosthesis 100 includes a distal portion 114 extending from a distal end 110. The distal portion 114 can be defined by a first material structure 302. The first material structure 302, the second material structure 322, and the fenestration tubes 104 form a tubular body 102. As described previously, the fenestration tubes 104 can be separate materials from the first material structure 302 and the second material structure 322, or can be openings, pockets, passageways, or the like formed from the first material structure 302 and the second material structure 322. The first material structure 302 and the second material structure 322 define a bore 106 of the tubular body 102. The first material structure 302 and the second material structure 322 are illustrated as including two material layers; however, any number of layers may be included in the first material structure 302 and the second material structure 322. A wire stent 122 may circumferentially surround the first material structure 302 and the second material structure 322 of the fenestrated endovascular prosthesis 100.

As illustrated in 4A through 4C, a proximal opening 210 of each of the fenestration tubes 104 may be proximal to a proximal edge 320 of the first material structure 302. A distal opening 212 of each of the fenestration tubes 104 may be distal to a distal edge 324 of the second material structure 322. This ensures that the lumen 206 of each of the fenestration tubes 104 is open to a bore 106 of the tubular body 102 and an exterior of the fenestrated endovascular prosthesis 100, without being blocked by the first material structure 302 and/or the second material structure 322. Further, although the fenestration tubes 104 are illustrated as being disposed at the same positions along the length of the tubular body 102, in some examples, the fenestration tubes 104 may be disposed at different positions along the length of the tubular body 102. This may allow for better positioning of the distal openings 212 of the fenestration tubes 104 relative to branched arteries into which secondary endovascular prostheses are to be deployed from the distal openings 212 of the fenestration tubes 104.

FIGS. 4A through 4C illustrate the fenestrated endovascular prosthesis 100 as including four fenestration tubes 104. However, more or fewer fenestration tubes 104 may be included. Further, the fenestration tubes 104 are illustrated as being evenly dispersed around the periphery of the first material structure 302, with each of the fenestration tubes 104 being radially spaced apart from adjacent fenestration tubes 104 by about 90°. However, any suitable spacing between adjacent fenestration tubes 104 may be provided. For example, all four fenestration tubes 104 may be provided on one half of the fenestrated endovascular prosthesis 100; two sets of two of the fenestration tubes 104 may be spaced relatively close to one another and relatively far from the other set of two fenestration tubes 104; or the like. The fenestration tubes 104 may be rotationally spaced apart from one another depending on a layout of an artery in which the fenestrated endovascular prosthesis 100 is to be deployed and the rotational spacing of branched arteries extending from that artery. In other words, the fenestration tubes 104 may be configured and rotationally spaced to align with branched arteries extending from a damaged artery when the fenestrated endovascular prosthesis 100 is deployed in a patient's body.

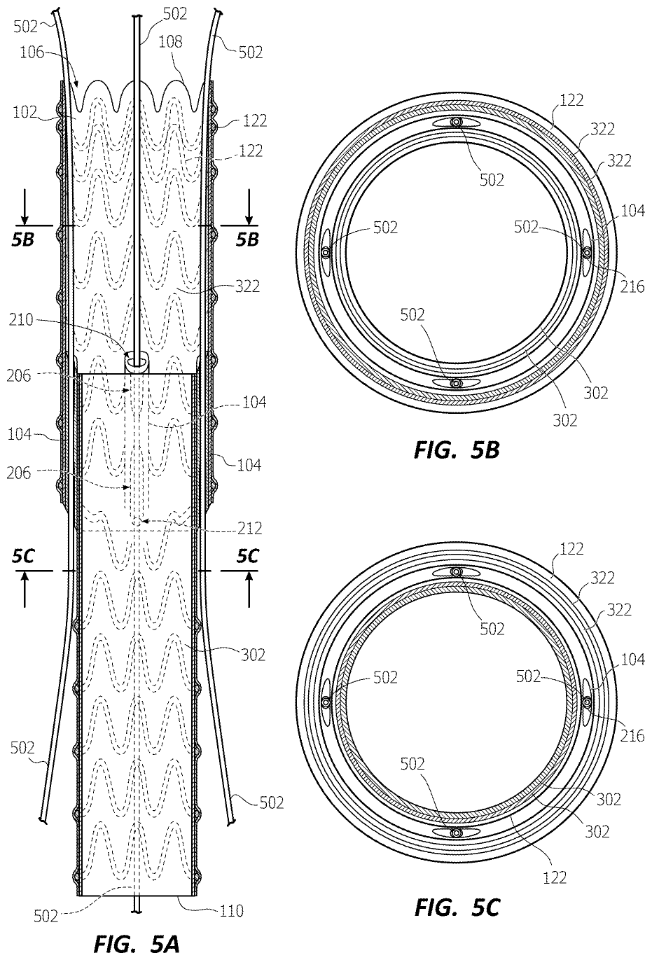

In FIGS. 5A through 5C, guidewires 502 are positioned in each of the fenestration tubes 104. The guidewires 502 may be placed in the fenestration tubes 104 to guide subsequently inserted devices into the fenestration tubes 104. For example, a fenestration dilator, examples of which are discussed in detail below, may be guided along a respective guidewire 502 to transition a fenestration tube 104 through which the guidewire 502 extends from the scaled configuration to the open configuration. After the fenestration tube 104 is un-scaled to the open configuration, a secondary endovascular prosthesis may be guided along the guidewire 502 and deployed in the fenestration tube 104 to provide a pathway for blood to flow from the bore 106 of the fenestrated endovascular prosthesis 100 to an exterior of the fenestrated endovascular prosthesis 100, such as to a branched artery or the like.

The guidewires 502 can extend from outside the fenestrated endovascular prosthesis 100 (such as along the first material structure 302 of the distal portion 114 of the fenestrated endovascular prosthesis 100), through the fenestration tubes 104, through a proximal portion 112 of the fenestrated endovascular prosthesis 100 (such as along the second material structure 322), and out of the proximal end 108 of the fenestrated endovascular prosthesis 100. As illustrated in FIGS. 5A through 5C, the seal regions 216 may be included in the fenestration tubes 104, even after the guidewires 502 are positioned in the fenestration tubes 104. As such, the fenestration tubes 104 may remain in the sealed configuration after the guidewires 502 are positioned within the fenestration tubes 104. The guidewires 502 may have sufficiently small diameters such that the guidewires 502 do not un-seal the seal regions 216 of the fenestration tubes 104. This may result in the seal regions 216 remaining sealed, even after the guidewires 502 are removed from the fenestration tubes 104. The guidewires 502 can have diameters in a range from about 0.005 inches to about 0.020 inches, such as about 0.010 inches, which prevents the guidewires 502 from un-scaling the seal regions 216. This prevents leakage of blood from the fenestration tubes 104 when the fenestrated endovascular prosthesis 100 is deployed, both when the guidewires 502 are positioned within the fenestration tubes 104 and when the guidewires 502 are removed from the fenestration tubes 104.

In some examples, the guidewires 502 can be positioned within the fenestration tubes 104 prior to the fenestrated endovascular prosthesis 100 being deployed within a patient's body. For example, the guidewires 502 can be positioned in each of the fenestration tubes 104, and the fenestrated endovascular prosthesis 100 can be crimped into a deployment device, discussed in detail below. The guidewires 502 may extend through the deployment device. Thus, the guidewires 502 may be part of the fenestrated endovascular prosthesis 100 when it is deployed into the patient's body. In some examples, the guidewires 502 may be fed through the patient's body before the fenestrated endovascular prosthesis 100 is deployed. For example, the guidewires 502 may be advanced from an insertion site (e.g., a femoral, jugular, or other insertion site), past a treatment site, to an extraction site (e.g., a femoral, jugular, or other extraction site). The fenestrated endovascular prosthesis 100 and the guidewires 502 can then be advanced such that the fenestrated endovascular prosthesis is deployed at the treatment site.

In some examples, the fenestrated endovascular prosthesis 100 can be deployed without guidewires 502 being positioned within the fenestration tubes 104. The guidewires 502 can be advanced into respective fenestration tubes 104 after the fenestrated endovascular prosthesis 100 is deployed. The guidewires 502 can be advanced into respective proximal openings 210 or distal openings 212 of the fenestration tubes 104, through the seal regions 216, and out of the other of the respective proximal openings 210 or distal openings 212 of the fenestration tubes 104. In some examples, radiopaque markers, which may be the same as or similar to the radiopaque marker bands 214, discussed above with respect to FIGS. 2A and 2B, can be disposed adjacent the proximal openings 210 and/or the distal openings 212 to facilitate access of the guidewires 502 into the fenestration tubes 104 using fluoroscopy or the like.

In some examples, the guidewires 502 can extend partially through the fenestration tubes 104 prior to the fenestrated endovascular prosthesis 100 being deployed. For example, the guidewires 502 can be disposed within the fenestration tubes 104 adjacent the seal regions 216, or can extend partially through the seal regions 216. The guidewires 502 can be advanced through the seal regions 216 after the fenestrated endovascular prosthesis 100 is deployed at a treatment site in a patient's body. In some examples, the guidewires 502 can be used to advance treatments to the fenestration tubes 104 without the guidewires 502 extending completely through the fenestration tubes 104. In various examples, none, some, or all of the guidewires 502 may be advanced through the fenestration tubes 104.

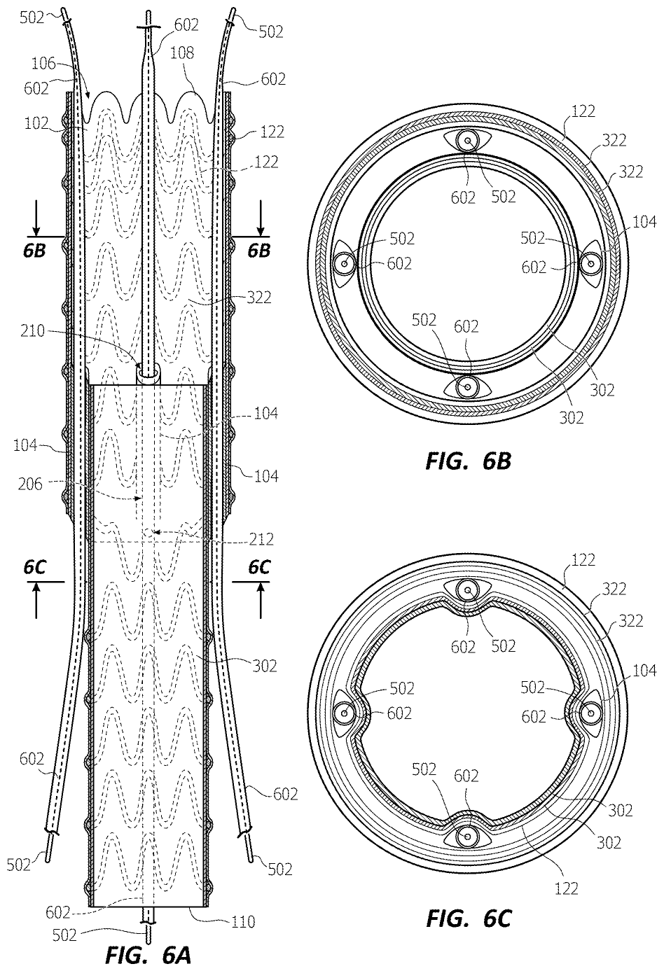

In FIGS. 6A through 6C, fenestration dilators 602 are positioned in each of the fenestration tubes 104. The fenestration dilators 602 may be used to transition the fenestration tubes 104 from the scaled configuration to the open configuration. The fenestration dilators 602 may be advanced along the guidewires 502 into the fenestration tubes 104. In the example illustrated in FIGS. 6A through 6C, the fenestration dilators 602 may be advanced along the guidewires 502 and through the fenestration tubes 104 in a direction from the distal openings 212 to the proximal openings 210. In some examples, the fenestration dilators 602 may be advanced along the guidewires 502 and through the fenestration tubes 104 in a direction from the proximal openings 210 to the distal openings 212.

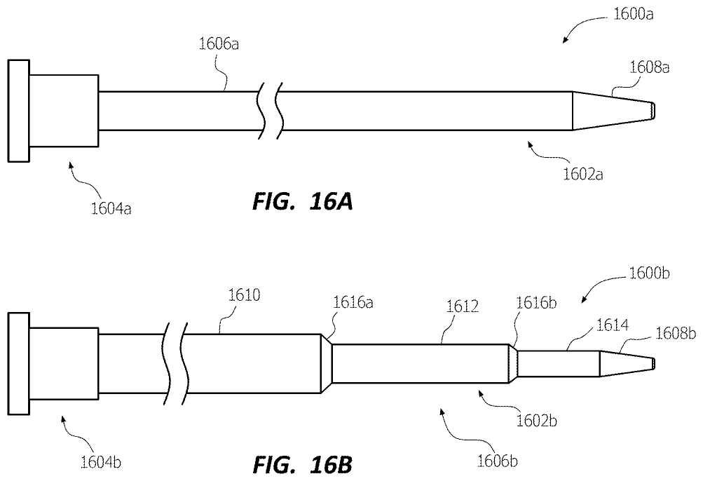

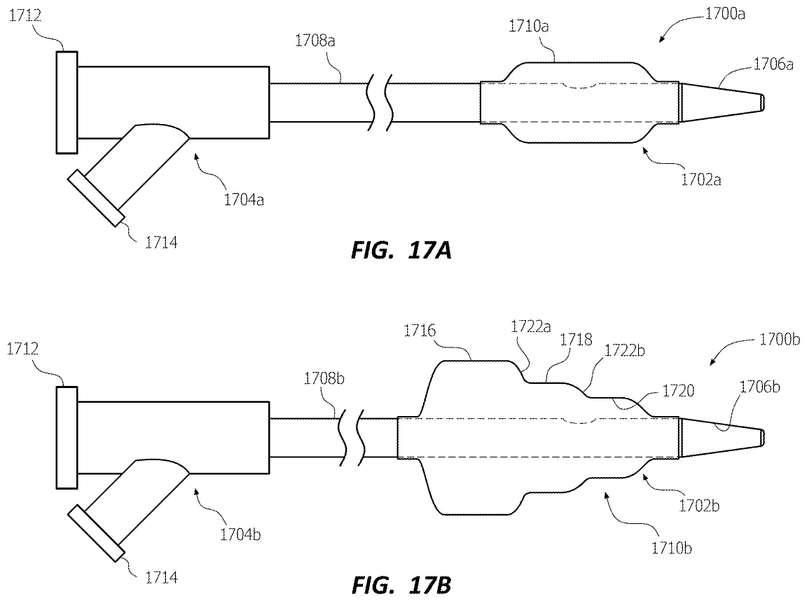

Each respective fenestration tube 104 may be un-sealed from the sealed configuration to the open configuration by advancing a fenestration dilator 602 along a guidewire 502 and into a lumen 206 of the fenestration tube 104. The fenestration dilator 602 is pressed against a seal region 216 of the fenestration tube 104 in the lumen 206 and a force is applied to un-seal or selectively open the seal region 216. In some examples, the fenestration dilators 602 may be tapered dilators (discussed below with respect to FIGS. 16A and 16B), which expand the seal region 216 as a tapered portion of the fenestration dilator 602 extends through the seal region 216. In some examples, the fenestration dilators 602 may be balloon dilators (discussed below with respect to FIGS. 17A and 17B), which are inserted into the seal region 216 and expand the seal region 216 by expanding the diameter of the fenestration dilator 602. In some examples, the fenestration dilator 602 may include a medical instrument capable of cutting the seal region 216, which may be used to form a slit in the seal region 216. Various examples of fenestration dilators 602 that may be used to un-seal the seal regions 216 of the fenestration tubes 104 are discussed in detail below.

Although FIGS. 6A through 6C illustrate all of the fenestration tubes 104 of the fenestrated endovascular prosthesis 100 as being opened by the fenestration dilators 602, in some examples, only selected ones of the fenestration tubes 104 may be opened. For example, only selected fenestration tubes 104 in which secondary endovascular prostheses are to be deployed may be opened. In an example in which secondary endovascular prostheses are to be deployed from the fenestrated endovascular prosthesis 100 to a pair of renal arteries, two of the fenestration tubes 104 disposed proximal to the renal arteries may be opened by the fenestration dilators 602. Un-scaling the seal region 216 can allow blood to flow from the bore 106 of the fenestrated endovascular prosthesis 100 out through the fenestration tubes 104. Thus, fenestration tubes 104 that are not to be used to deploy secondary endovascular prostheses may be left in the sealed configuration in order to prevent undesired blood flow from the fenestrated endovascular prosthesis 100.

Thus, each of the fenestration tubes 104 can be selectively un-sealed to the open configuration for use, or left in the sealed configuration by a practitioner during treatment. This allows for the fenestrated endovascular prosthesis 100 to be configurable based on patient-specific needs and reduces variations of the fenestrated endovascular prosthesis 100 needed to treat patients. In some examples, the fenestrated endovascular prosthesis 100 can be deployed in a vessel without branched arteries, and each of the fenestration tubes 104 can be left in the sealed configuration. In such an example, the fenestrated endovascular prosthesis 100 functions as a non-fenestrated vascular prosthesis or stent. After selected ones of the fenestration tubes 104 are un-sealed into the open configuration, the fenestration dilators 602 can be used to advance other therapies, such as secondary endovascular prostheses (discussed below with respect to FIGS. 7A through 7C), or can be removed through an extraction site (e.g., a femoral, jugular, or other extraction site).

In FIGS. 7A through 7C, secondary endovascular prostheses 702 are positioned in each of the fenestration tubes 104. The secondary endovascular prostheses 702 may be used to provide pathways from the bore 106 of the fenestrated endovascular prosthesis 100, through the fenestration tubes 104, to an exterior of the fenestrate fenestrated endovascular prosthesis 100. For example, a respective secondary endovascular prosthesis 702 may provide a blood flow path from the bore 106 of the fenestrated endovascular prosthesis 100, through the fenestration tubes 104, to a branched artery outside the fenestrated endovascular prosthesis 100. The secondary endovascular prostheses 702 may be sealed in both the fenestration tubes 104 and the branched arteries to which the secondary endovascular prostheses 702 extend.

A particular secondary endovascular prosthesis 702 can be deployed in a respective fenestration tube 104 and a branched artery by advancing a secondary deployment device from a proximal vascular access site (e.g., a jugular access site) along a respective guidewire 502. The guidewire 502 may be fed through a secondary distal tip of the secondary deployment device, through the secondary endovascular prosthesis 702, and through the secondary deployment device as the secondary deployment device is advanced to the fenestrated endovascular prosthesis 100. The secondary deployment device may be advanced proximally along the guidewire 502 such that the secondary distal tip is advanced through the proximal end 108 of the fenestrated endovascular prosthesis 100, into the bore 106 of the fenestrated endovascular prosthesis 100, through the proximal opening 210 of the fenestration tube 104, through the lumen 206 of the fenestration tube 104, and out of the distal opening 212 of the fenestration tube 104. The guidewire 502 may then be removed through the proximal vascular access site or through a distal vascular access site (e.g., a femoral access site).

The secondary deployment device may then be advanced via the proximal access site such that the secondary distal tip of the secondary deployment device extends into the branched artery. In some examples, both the proximal and distal ends of the secondary deployment device can be configured with atraumatic ends or ends that can be guided through the anatomy. In some examples, the guidewire 502 may be pulled through the proximal vascular access site until the guidewire 502 reaches the distal opening 212 of the fenestration tube 104, advanced into the branched artery, and the secondary deployment device may be advanced along the guidewire 502 into the branched artery. In some examples, a secondary guidewire may be connected to the guidewire 502, the guidewire 502 can be pulled through the distal vascular access site until the secondary guidewire extends through the distal opening 212 of the fenestration tube 104, the secondary guidewire can be disconnected from the guidewire 502 and the guidewire 502 removed through the distal vascular access site, the secondary guidewire can be advanced into the branched artery, and the secondary deployment device may be advanced along the secondary guidewire into the branched artery. In some examples, the secondary deployment device can be advanced and deployed through the distal access site.

Once the secondary deployment device is positioned at a desired location within the fenestration tube 104 and the branched artery, the secondary endovascular prosthesis 702 is deployed from the secondary deployment device. The secondary endovascular prosthesis 702 may be positioned within a secondary deployment device in a crimped or delivery configuration. The secondary endovascular prosthesis 702 can be deployed to provide a bridge for blood flow between the bore 106 of the fenestrated endovascular prosthesis 100, through the fenestration tube 104, into the branched artery. The secondary deployment device may be similar to the deployment device discussed below with respect to FIGS. 8 through 14E, and can be configured to selectively deploy the secondary endovascular prosthesis 702 based on input provided by a practitioner outside a patient's body. The secondary endovascular prosthesis 702 may be configured to be expanded by a balloon or may be self-expanding.

As illustrated in FIG. 7A, a proximal end 704 of a particular secondary endovascular prosthesis 702 can be positioned proximal the proximal opening 210 of a respective fenestration tube 104, and a distal end distal end 706 of the secondary endovascular prosthesis 702 can be positioned distal the distal opening 212 of the fenestration tube 104. This provides for maximal sealing between the secondary endovascular prosthesis 702 and the fenestration tube 104. Moreover, the secondary endovascular prostheses 702 can include wire stents, which may be similar to or the same as the wire stents 122, discussed above. The secondary endovascular prostheses 702 may hold the fenestration tubes 104 open in the open configuration, and positioning the secondary endovascular prostheses 702 with proximal ends 704 extending proximal to the proximal openings 210 ensures that the entire lengths of the fenestration tubes 104 are in the open configuration. In some examples, the proximal end 704 of the secondary endovascular prosthesis 702 can be positioned distal to or even with the proximal opening 210 of the fenestration tube 104. The proximal ends 704 of the secondary endovascular prostheses 702 may be within about 0.05 mm to about 2.0 mm of the proximal openings 210 of the fenestration tubes 104. The secondary endovascular prostheses 702 and the fenestration tubes 104 may include radiopaque marker bands or the like, which may be used in conjunction with radiography, fluoroscopy, or the like to confirm relative positions of the secondary endovascular prostheses 702 and the fenestration tubes 104 before the secondary endovascular prostheses 702 are deployed in the fenestration tubes 104.

In some examples, the secondary endovascular prosthesis 702 can include a wire stent (which may be similar to or the same as the wire stent 122 provided on the fenestrated endovascular prosthesis 100 and discussed above with respect to FIGS. 1 and 3D). The wire stent of the secondary endovascular prosthesis 702 can cause a tubular body of the secondary endovascular prosthesis 702 to be pressed against fenestration tube walls of a fenestration tube 104 and walls of a branched artery in which the secondary endovascular prosthesis 702 is deployed by a spring force of the wire stent. This seals the secondary endovascular prosthesis 702 to the fenestration tube walls of the fenestration tube 104 and the walls of the branched artery, which forces blood to flow from the bore 106 of the tubular body, through the secondary endovascular prosthesis 702, into the branched artery. As such, the fenestrated endovascular prosthesis 100 and secondary endovascular prostheses 702 deployed in the fenestrated endovascular prosthesis 100 are used to bypass a damaged section of an artery, even in cases in which branched arteries split from the damaged section of the artery.

After the secondary endovascular prosthesis 702 is deployed, the secondary deployment device and any guidewires, such as guidewires 502 or secondary guidewires, can be removed from the patient's body through the proximal vascular access site and/or the distal vascular access site.

FIG. 8 is a perspective view of a deployment device 800 that can be configured to deploy a fenestrated endovascular prosthesis 100, including guidewires 502 extending therethrough, at a treatment site within a patient. The deployment device 800 comprises a handle assembly 802 adjacent a proximal end of the deployment device 800. An elongate delivery catheter assembly 804 extends distally from the handle assembly 802 to a distal tip 806 (also referred to as a delivery tip). The handle assembly 802 may provide a proximal user input, and may include one or more components configured to allow a practitioner to deploy or otherwise manipulate the fenestrated endovascular prosthesis 100 disposed within the delivery catheter assembly 804.

In use, the handle assembly 802 may be disposed outside of a patient's body, while the delivery catheter assembly 804 is advanced to a treatment site within the patient's body. For example, the delivery catheter assembly 804 may be advanced from an insertion site (e.g., a femoral or jugular insertion site) to the treatment site within the patient's vasculature. The delivery catheter assembly 804 may be configured to be advanced through bends, turns, or other structures within the anatomy of the vasculature. The fenestrated endovascular prosthesis 100 may be disposed within a portion of the delivery catheter assembly 804 such that a practitioner may deploy the fenestrated endovascular prosthesis 100 from a distal end of the delivery catheter assembly 804 through manipulation of the proximal user input of the handle assembly 802.

The deployment device 800 can be configured to slidably receive the guidewires 502. As discussed previously, the guidewires 502 may be provided in the fenestrated endovascular prosthesis 100 to provide access to fenestration tubes of the fenestrated endovascular prosthesis 100 after the fenestrated endovascular prosthesis 100 is deployed. For example, the guidewires 502 may allow a practitioner to un-seal the fenestration tubes from a sealed configuration to an open configuration (for example, by advancing a dilator along the guidewire 502), to position and deploy secondary endovascular prostheses in the fenestration tubes, and the like.

As illustrated in FIG. 8, the guidewires 502 may extend from outside a periphery of the distal tip 806 into the delivery catheter assembly 804. The guidewires 502 may extend through the delivery catheter assembly 804, the fenestrated endovascular prosthesis 100, and the handle assembly 802, and may exit a proximal end of the handle assembly 802. Channels, grooves, and the like may be provided in the various components of the deployment device 800, which allow for the guidewires 502 to slidably extend through the deployment device 800. In use, the guidewires 502 and a deployment guidewire (not separately illustrated) may be advanced through a patient's body, from the insertion site to the extraction site. The deployment device 800 may be advanced along the deployment guidewire and the guidewires 502 may also be advanced until the fenestrated endovascular prosthesis 100 is positioned at the treatment site. The fenestrated endovascular prosthesis 100 may be deployed, the deployment device 800 may be withdrawn along the guidewires 502 and removed from the insertion site. The deployment guidewire may be removed from the insertion site or the extraction site once the fenestrated endovascular prosthesis 100 is positioned at the treatment site, before or after the deployment device 800 is removed. Providing channels, grooves, and the like in the deployment device 800 for allowing the guidewires 502 to slidably extend through the deployment device 800 allows for the fenestrated endovascular prosthesis 100 to be deployed with the guidewires 502 in place in the fenestration tubes. This provides access to the fenestration tubes for deploying secondary endovascular prostheses, and aids in providing blood flow pathways between the fenestrated endovascular prosthesis 100 and branched arteries.

In some examples, a practitioner can gain access to a treatment site in the vasculature through both the insertion site and the extraction site. The practitioner can advance an insertion sheath from the insertion site and an extraction sheath from the extraction site some distance, such as advancing the insertion sheath and the extraction sheath up to or adjacent to the treatment site, with the treatment site between the insertion sheath and the extraction sheath. The practitioner can advance an extraction snare through the extraction site and the extraction sheath, and the extraction snare can be advanced into the insertion sheath. An insertion snare can be advanced from the insertion site into the insertion sheath, and can be used to capture a distal end of the extraction snare. The extraction snare can be used to capture the deployment guidewire and/or the guidewires 502, and can be used to advance the deployment guidewire and/or the guidewires 502 from the insertion site or a point between the insertion site and the extraction site through the extraction site. The extraction snare can be used to advance the guidewires 502 as the deployment device 800 is advanced to the treatment site. The guidewires 502 can be sufficiently long that the guidewires 502 extend from the insertion site out through the extraction site before the deployment device 800 is inserted, the guidewires 502 are advanced with the deployment device 800 as the deployment device 800 is advanced to the treatment site, and the guidewires 502 extend distally out of the insertion site after the deployment device 800 is advanced to the treatment site. The guidewires 502 may not move relative to the deployment device 800 as the deployment device 800 is advanced to the treatment site. After the fenestrated endovascular prosthesis 100 is deployed, the deployment device 800 may be removed while the guidewires 502 remain in place, extending from the insertion site through the treatment site and out of the extraction site.

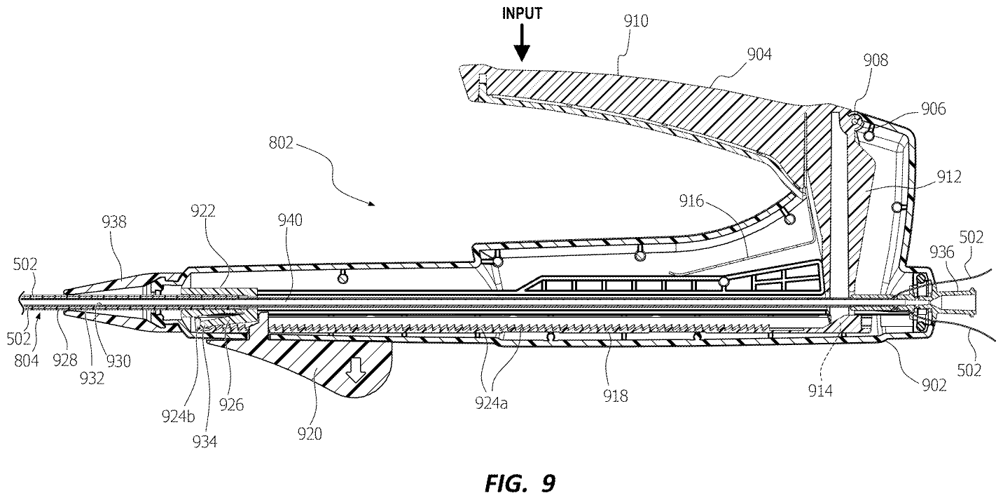

FIG. 9 is a cross-sectional view of a portion of the deployment device 800 of FIG. 8. Specifically, FIG. 9 is a side view of a portion of the deployment device 800 of FIG. 8, taken through a cross-sectional plane extending vertically and intersecting a longitudinal axis of the deployment device 800, when the deployment device 800 is positioned as shown in FIG. 8. The longitudinal axis of the deployment device 800 extends along the center of the delivery catheter assembly 804, including along the center of components of the delivery catheter assembly 804 that overlap with the handle assembly 802, such as an inner sheath 930 and an intermediate sheath 932, as shown in FIG. 9.

The handle assembly 802 is configured to be grasped or otherwise manipulated by a user, such as a medical practitioner, and is configured to provide user input to the delivery catheter assembly 804. The delivery catheter assembly 804 is configured to extend to a treatment site within a patient's body and is configured to deploy a fenestrated endovascular prosthesis 100 at the treatment site. A longitudinal axis of the delivery catheter assembly 804 extends in a distal direction away from the handle assembly 802. The proximal direction opposite the distal direction correlates to a direction defined along the longitudinal axis of the delivery catheter assembly 804 and extending from the distal tip 806 (illustrated in FIG. 8) toward the handle assembly 802.

FIG. 9 depicts various internal components of the handle assembly 802, exposed by the cross-sectional view. A portion of the delivery catheter assembly 804 is shown extending from the handle assembly 802. The handle assembly 802 comprises a housing 902. The housing 902 surrounds certain components of the handle assembly 802, as shown, and provides a grip surface for a practitioner.

The housing 902 is operably coupled to an actuator 904. Manipulation of the actuator 904 with respect to the housing 902 may be configured to deploy a fenestrated endovascular prosthesis 100, as described in further detail below. In the example of FIG. 9, the actuator 904 is rotatably coupled to the housing 902 by a pin 906. The pin 906 extends from the housing 902 and may be integrally formed with one or more other portions of the housing 902. As shown, the pin 906 extends through a pin aperture 908 in the actuator 904.

Other arrangements for operably coupling the actuator 904 and the housing 902 are within the scope of this disclosure. For example, the pin 906 may be integral with a portion of the actuator 904 and may be received in an opening, sleeve, or aperture formed in the housing 902. Other types of designs of rotatable couplings, including a separate coupling component such as a hinge are within the scope of this disclosure. Still further, a compliant mechanism, such as a deformable flange, may be utilized to rotatably couple the actuator 904 and the housing 902, including compliant couplings integrally formed with the actuator 904, the housing 902, or both. It is within the scope of this disclosure to slidably couple an actuator (such as the actuator 904) to a housing (such as the housing 902). Configurations in which the actuator 904 is manipulated through rotation, translation, or other displacement relative to the housing 902 are all within the scope of this disclosure.

The actuator 904 includes an input portion 910 extending from the pin aperture 908. In the example of FIG. 9, the input portion 910 comprises a surface, at least partially exposed with respect to the housing 902. In operation, a user may manipulate the actuator 904 by exerting an input force on the input portion 910, illustrated by the arrow labeled “input” in FIG. 9, displacing the input portion 910 generally toward the longitudinal axis of the deployment device 800 (e.g., in an input direction) and causing the actuator 904 to rotate about the pin 906 with respect to the housing 902. Displacement of the actuator 904 due to the input force corresponds to depression of the actuator 904 or depression of the actuator 904 with respect to the housing 902.

The actuator 904 may further comprise a transfer arm 912 extending from the pin aperture 908. The transfer arm 912 may be rigidly coupled to the input portion 910, including examples wherein both the transfer arm 912 and the input portion 910 are integrally formed with the rest of the actuator 904. The transfer arm 912 extends to a ratchet slide-engaging portion 914. Depression of the input portion 910 in the input direction displaces the transfer arm 912 as the actuator 904 is rotated about the pin 906.

Depression of the input portion 910 thus causes displacement of the ratchet slide-engaging portion 914 with respect to the housing 902. This displacement of the ratchet slide-engaging portion 914 can be understood as rotation about the pin 906 having a proximal translation component and a vertical translation component. In other words, rotation of the input portion 910 in the in the input direction will displace the ratchet slide-engaging portion 914 both proximally and vertically with respect to the housing 902.

A spring 916 may be disposed between the actuator 904 and the housing 902. The spring 916 may be configured to resist displacement of the actuator 904 in the input direction and may be configured to return the actuator 904 to the relative position shown in FIG. 9 after the input portion 910 of the actuator 904 has been depressed by a user. When the handle assembly 802 is unconstrained, the spring 916 may thus maintain (or return to) the relative position of the actuator 904 with respect to the housing 902 as shown in FIG. 9. As the actuator 904 is depressed with respect to the housing 902, the spring 916 compresses and the ratchet slide-engaging portion 914 is displaced as described above. Again, the displacement of the ratchet slide-engaging portion 914 with respect to the housing 902 can be understood as having a proximal component and a vertical component.

The ratchet slide-engaging portion 914 may be operably coupled to a ratchet slide 918 such that displacement of the ratchet slide-engaging portion 914 displaces the ratchet slide 918. The ratchet slide 918 may be constrained such that the ratchet slide 918 is configured for proximal or distal displacement with respect to the housing 902, but is not configured for vertical displacement with respect to the housing 902. Thus, operable coupling of the ratchet slide-engaging portion 914 to the ratchet slide 918 may allow for sliding interaction between the ratchet slide-engaging portion 914 and the ratchet slide 918, with the proximal or distal component of the displacement of the ratchet slide-engaging portion 914 being transferred to the ratchet slide 918. In other words, the ratchet slide 918 may be displaced in a direction parallel to the longitudinal axis of the deployment device 800 in response to the input portion 910 of the actuator 904 being displaced in the input direction at an angle to the longitudinal axis of the deployment device 800.

In the example of FIG. 9, a safety member 920 may prevent proximal displacement of the ratchet slide 918. Discussion herein relating to displacement of the ratchet slide 918 and related components may be understood as disclosure relevant to a configuration of the handle assembly 802 in which the safety member 920 has been removed. The safety member 920 may be configured with a circular or partially circular opening configured to snap onto an outside surface of a portion of the deployment device 800. The safety member 920 may comprise a safety lug that extends through a ratchet slide safety opening of the ratchet slide 918 and a similar safety opening in the housing 902 (not separately illustrated). When the safety lug is disposed within these openings, the safety lug may prevent proximal displacement of the ratchet slide 918. This prevents inadvertent deployment of the fenestrated endovascular prosthesis 100. A user may leave the safety member 920 in place during displacement of the delivery catheter assembly 804 to a treatment site. Due to interactions between the ratchet slide 918 and actuator 904, the safety member 920 prevents displacement of the actuator 904 while the safety lug extends through the openings in the ratchet slide 918 and the housing 902. In some examples, the safety member 920 may be tethered to the deployment device 800, or may comprise a sliding switch or other element operably coupled to the housing 902 or other components of the deployment device 800.

As the actuator 904 is depressed with respect to the housing 902, the ratchet slide 918 may be proximally displaced with respect to the housing 902. One or both of the ratchet slide 918 and actuator 904 may interact with the housing 902 such that there is a positive stop to arrest the depression of the actuator 904 and/or proximal displacement of the ratchet slide 918. This positive stop may be an engaging ledge, shoulder, lug, detent, or other feature coupled to the housing 902, including features integrally formed on the housing 902. As depicted, the positive stop can be disposed proximally of a proximal end of the ratchet slide 918. For example, the proximal end of the ratchet slide 918 can interact with a portion of the housing 902 (e.g., a ledge, shoulder, or the like) disposed proximally of the proximal end of the ratchet slide 918. Accordingly, the handle assembly 802 may be configured such that the ratchet slide 918 is displaced or “travels” as much as possible during depression of the actuator 904.

A full stroke of the actuator 904 may correspond to displacement from the unconstrained position shown in FIG. 9, to the positive stop caused by interaction with the housing 902 when the actuator 904 is depressed. A partial stroke of the actuator 904 may correspond to displacement from the unconstrained position shown in FIG. 9, to each and/or any position prior to the positive stop caused by interaction with the housing 902 when the actuator 904 is depressed. Release of the actuator 904 following a full stroke or a partial stroke may result in a return of the actuator 904 to the unconstrained position, due to the biasing force provided by the spring 916. The unconstrained position shown in FIG. 9 refers to lack of constraint due to user input. In this state, the spring 916 may be partially compressed, and interaction between the actuator 904 and the housing 902 may prevent rotation of the actuator 904 about the pin 906 in the opposite direction to depression of the actuator 904, or the return direction. In other words, interaction between the actuator 904 and the housing 902 (or features of the housing 902) may create a positive stop to the return motion of the actuator 904 as well.

The ratchet slide 918 may be proximally displaced during depression of the actuator 904. Such displacement may correspond to a configuration in which the safety member 920 has been removed. Proximal displacement of the ratchet slide 918 may proximally displace a carrier 922 due to interaction between one or more carrier-engaging ratchet lugs 924a on the ratchet slide 918 and a ratchet slide-engaging arm 926 coupled to the carrier 922. In some examples, the carrier 922 may be coupled to an outer sheath 928 of the delivery catheter assembly 804. For example, the carrier 922 may be fixedly and/or rigidly coupled to the outer sheath 928. In some examples, an inner sheath 930 and/or an intermediate sheath 932 of the delivery catheter assembly 804 may be coupled to the handle assembly 802. For example, the inner sheath 930 and/or the intermediate sheath 932 may be fixedly and/or rigidly coupled to the handle assembly 802.

The distal tip 806 of the delivery catheter assembly 804 may be coupled to and/or integrally formed with the inner sheath 930. The distal tip 806 may comprise a flexible material and may be configured to be atraumatic. The distal tip 806 may comprise nylons, including PEBAX® polyether block amides.

A lumen 940 may extend along the inner sheath 930 from the proximal end of the deployment device 800 to the distal tip 806. A luer fitting 936 coupled to the housing 902 may be in communication with the lumen 940. A deployment guidewire may be placed in the distal tip 806 to guide the delivery catheter assembly 804 to a treatment site. The deployment guidewire may extend through the distal tip 806, the lumen 940, and the luer fitting 936. Fluid introduced into the luer fitting 936 may be utilized to flush the lumen 940, or may be delivered through the lumen 940 and the distal tip 806.

The inner sheath 930 may be fixed to the housing 902. For example, the proximal end of the inner sheath 930 may be fixed to the housing 902. The intermediate sheath 932 may also be fixed to the housing 902, and may extend over a portion of the inner sheath 930. The intermediate sheath 932 and inner sheath 930 may or may not be directly fixed to each other. In some examples, the intermediate sheath 932 may be a close slip fit over the inner sheath 930.

In some instances braided or coil reinforcements may be added to the outer sheath 928, the intermediate sheath 932, and/or the inner sheath 930 to increase kink resistance and/or elongation. Reinforcing members may comprise stainless steel, nitinol, or other materials and may be round, flat, rectangular in cross section, and so forth.

One, two, or all of the outer sheath 928, the intermediate sheath 932, and/or the inner sheath 930 may be configured with varying durometers or other properties along the length thereof. In some instances the outer sheath 928 may be configured with a proximal section with a durometer between 72 and 100 on the Shore D scale or may be greater than 100 on the Shore D scale. A second portion of the outer sheath 928 (e.g., an intermediate section) may have a durometer of 63 on the Shore D scale and a distal section of the outer sheath 928 may have a durometer between 40 and 55 on the Shore D scale. Any of these values, or the limits of any of the ranges, may vary by 15 units in either direction. In some examples, the distal section of the outer sheath 928 may extend distally from adjacent the distal tip 806 about 3 inches, the intermediate section of the outer sheath 928 may extend from the distal section to about 6 inches from the distal tip 806, and the proximal section of the outer sheath 928 may extend from the intermediate section to the housing 902. The proximal section, the intermediate section and the distal section of the outer sheath 928 may or may not correspond to the shaft section 1406, the flex zone 1404, and the pod 1402, respectively, as described above. The intermediate sheath 932 and/or the inner sheath 930 may be configured with varying durometer zones within the same ranges of hardness and length as the outer sheath 928.

Any of the inner sheath 930, the intermediate sheath 932, and the outer sheath 928 may have differing durometer or flex zones along their lengths, and these zones may overlap in various ways to create various stress/strain profiles for the overall delivery catheter assembly 804. Overlapping of such zones may reduce tendency to kink, including tendency to kink at transition zones. Further, the housing 902 may be coupled to a strain relief member 938 (as shown in FIG. 9).

The outer sheath 928, the intermediate sheath 932, and the inner sheath 930 may be formed of polymers, such as nylons, which may include polyether block amides or the like. During the manufacture of the outer sheath 928, the intermediate sheath 932, and/or the inner sheath 930, a “frosting” process may be performed by blowing air across the material during extrusion, or by using additives during the extrusion. This may result in the materials of the outer sheath 928, the intermediate sheath 932, and/or the inner sheath 930 having reduced friction, such as a low-friction outer surface.

In some examples, the distal tip 806 may be pulled into interference with the outer sheath 928 during manufacture, which pre-stresses the inner sheath 930 in tension. This may reduce any effects of material creep or elongation during sterilization, keeping the distal tip 806 snugly nested with the outer sheath 928. Further, an interface zone may be provided between the outer sheath 928 and the carrier 922 to provide tolerance during manufacture. This results in the outer sheath 928 being coupled to the carrier 922 at multiple points along an inside diameter of the carrier 922. The interface zone may enable manufacturing discrepancies or variations to be taken up during assembly to ensure a snug nest between the distal tip 806 and the outer sheath 928. The same interface zones and tolerance fit may be applied to the inner sheath 930 and/or the intermediate sheath 932 at interface zones between the housing 902 and each of the inner sheath 930 and/or the intermediate sheath 932. Specifically, interface zones may be provided along an inside diameter of the luer fitting 936 and the inner sheath 930 and/or the intermediate sheath 932.

In some examples, the outer sheath 928 and/or the handle assembly 802 may include indicia correlating to a degree to which a fenestrated endovascular prosthesis 100 has been deployed. These indicia may correspond to the position of the outer sheath 928 with respect to the housing 902, the inner sheath 930, and/or the intermediate sheath 932. For example, as the outer sheath 928 is drawn into the housing 902, different indicia may be exposed and/or covered to indicate the position of the outer sheath 928 and the degree to which the fenestrated endovascular prosthesis 100 has been deployed.

In some examples, the deployment device 800 may be configured such that the outer sheath 928 can be distally displaced after the fenestrated endovascular prosthesis 100 has been deployed. This allows for the distal tip 806 to be nested in the outer sheath 928 while the delivery catheter assembly 804 is withdrawn from a patient. Such configurations may include features of the handle assembly 802 that disengage the features of the carrier 922 from features of the housing 902 after deployment of the fenestrated endovascular prosthesis 100.

As illustrated in FIG. 9, the guidewires 502 may extend through the intermediate sheath 932, out of the intermediate sheath 932 and into the handle assembly 802, and out of the handle assembly 802 adjacent the luer fitting 936. Guides may be formed within the deployment device 800 such that the guidewires 502 slidably extend through various components of the deployment device 800. For example, guides may be provided in the intermediate sheath 932, and the guidewires 502 may slidably extend through the guides in the intermediate sheath 932. The guidewires 502 can be placed within the fenestrated endovascular prosthesis 100, the guidewires 502 can be inserted into the deployment device 800, and the fenestrated endovascular prosthesis 100 can be compressed and/or crimped and loaded into a pod of the deployment device 800. After the fenestrated endovascular prosthesis 100 is deployed by the deployment device 800, the guidewires 502 can move through the deployment device 800 as the deployment device is removed from or otherwise extracted from a patient's body. Positioning the guidewires 502 within the fenestrated endovascular prosthesis 100 prior to deployment within the patient's body aids in the deployment of secondary endovascular prostheses in fenestration tubes of the fenestrated endovascular prosthesis 100 after the fenestrated endovascular prosthesis 100 is deployed.

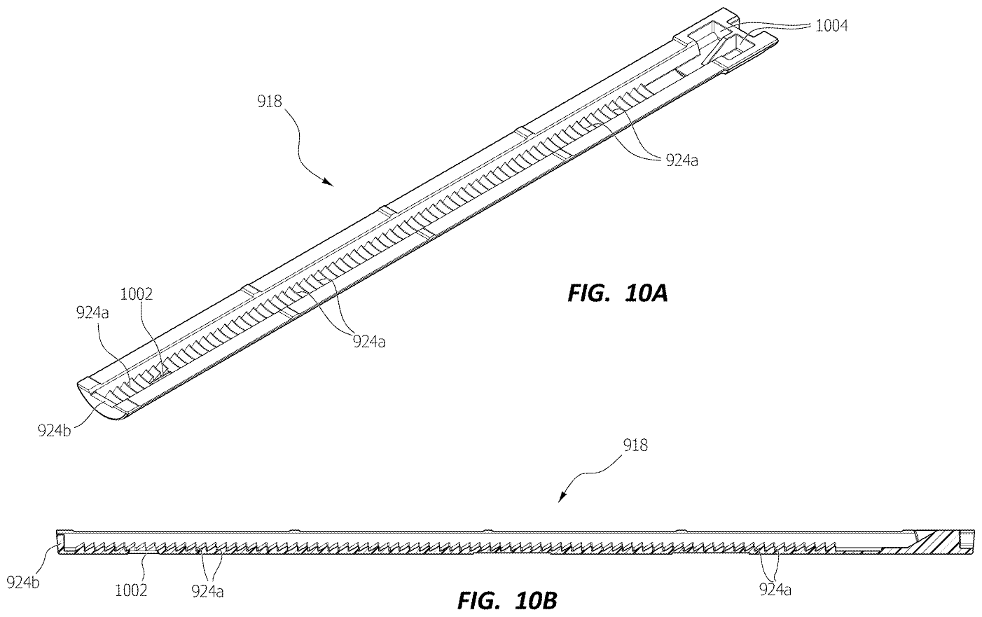

FIG. 10A is a perspective view of the ratchet slide 918 of the deployment device 800 of FIGS. 8 and 9. FIG. 10B is a cross-sectional view of the ratchet slide 918 of FIG. 10A, taken through a vertical plane disposed along a longitudinal centerline of the ratchet slide 918. When the ratchet slide 918 is disposed within the handle assembly 802 of FIG. 9, this cross-sectional plane would intersect the longitudinal axis of the deployment device 800.

As shown in FIGS. 9, 10A, and 10B, the ratchet slide 918 may include a plurality of carrier-engaging ratchet lugs 924a. The carrier-engaging ratchet lugs 924a may be spaced at intervals (e.g., at even intervals) along the longitudinal direction of the ratchet slide 918. The plurality of carrier-engaging ratchet lugs 924a may be disposed semi-continuously. For example, consecutive carrier-engaging ratchet lugs 924a may be spaced about 5 mm or less from each other, about 4 mm or less from each other, about 3 mm or less from each other, about 2 mm or less from each other, about 1 mm or less from each other, or any other suitable distance from each other. A distal-most carrier-engaging ratchet lug 924b may be denoted as reference numeral 924b.

The ratchet slide 918 further comprises a ratchet slide safety opening 1002 (configured to engage with the safety member 920) and an actuator-engaging opening 1004. These features are discussed in more detail below.

Interaction between the ratchet slide-engaging portion 914 of the actuator 904 and the ratchet slide 918 may proximally displace the ratchet slide 918 with respect to the housing 902. Engagement between the carrier 922 and one of the carrier-engaging ratchet lugs 924a may proximally displace the carrier 922 as the ratchet slide 918 is proximally displaced with respect to the housing 902. In the configuration of FIG. 9, the ratchet slide-engaging arm 926 of the carrier 922 is engaged with the distal-most carrier-engaging ratchet lug 924b.

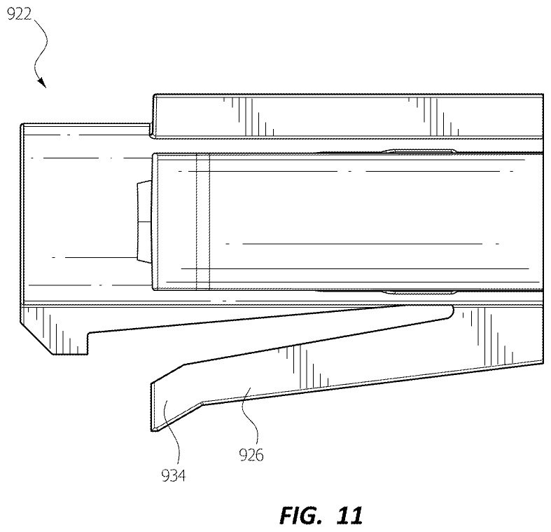

FIG. 11 is a side view of the carrier 922 of the deployment device 800 of FIGS. 8 and 9. As shown in FIG. 11, the ratchet slide-engaging arm 926 extends radially away from a longitudinal axis of the carrier 922. When the carrier 922 is disposed within the handle assembly 802, as illustrated in FIG. 9, the longitudinal axis of the carrier 922 is disposed along the longitudinal axis of the deployment device 800.

The ratchet slide-engaging arm 926 includes an angled portion 934 at a distal end of the ratchet slide-engaging arm 926. The angled portion 934 may extend radially away from the longitudinal axis of the carrier 922 at a greater angle than the ratchet slide-engaging arm 926. In some examples, the angled portion 934 can enhance engagement between the ratchet slide-engaging arm 926 and a respective carrier-engaging ratchet lug 924a. For example, as the carrier 922 moves in the proximal direction in response to the actuator being depressed, the angled portion 934 allows the ratchet slide-engaging arm 926 to deflect radially as the angled portion 934 contacts each of the respective carrier-engaging ratchet lugs 924a. When the actuator 904 is released, the carrier 922 may move in the distal direction, and the angled portion 934 contacts a respective carrier-engaging ratchet lug 924a to stop the distal movement of the carrier 922. The angled portion 934 can provide clearance for the ratchet slide-engaging arm 926, allowing the angled portion 934 to engage a respective carrier-engaging ratchet lug 924a (even when the carrier-engaging ratchet lugs 924a are closely spaced) without adjacent carrier-engaging ratchet lugs 924a interfering with the position of the ratchet slide-engaging arm 926 and preventing full engagement.

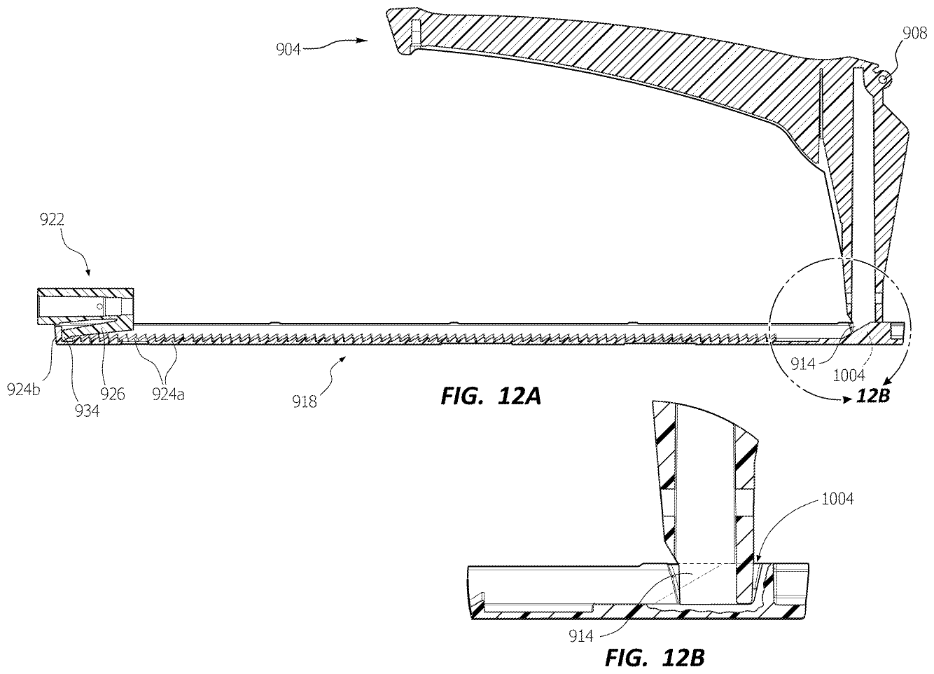

FIG. 12A is a cross-sectional view of a portion of the deployment device 800 shown in FIGS. 8 and 9. Specifically, the actuator 904, the ratchet slide 918, and the carrier 922 are shown in FIG. 12A in the same relative positions and along the same cross-sectional plane as in FIG. 9. FIG. 12B is a partial cut-away view of a portion of the cross-sectional view of FIG. 12A. In FIG. 12B, a portion of the ratchet slide 918 has been cut away in this view to show an engagement of the ratchet slide-engaging portion 914 with the intermediate sheath 932.

Referring to FIGS. 9 through 12B, when the actuator 904 is depressed with respect to the housing 902, the actuator 904 rotates around the pin aperture 908. This rotation causes displacement of the ratchet slide-engaging portion 914 of the actuator 904. The component of this displacement correlating to proximal displacement of the ratchet slide-engaging portion 914 proximally translates the ratchet slide 918. This proximal displacement is due to interaction between the ratchet slide-engaging portion 914 of the actuator 904 and the actuator-engaging opening 1004 of the ratchet slide 918. Stated another way, the walls or faces that define the actuator-engaging opening 1004 contact the ratchet slide-engaging portion 914 such that the ratchet slide 918 is displaced when the actuator 904 is displaced.

Proximal displacement of the ratchet slide 918 proximally displaces the carrier 922 due to interaction between the carrier-engaging ratchet lugs 924a and the ratchet slide-engaging arm 926. In the illustrated example, a distal surface of the angled portion 934 of the ratchet slide-engaging arm 926 is in contact with a proximal face of the distal-most carrier-engaging ratchet lug 924b. This contact exerts proximal force on the distal surface of the angled portion 934 of the ratchet slide-engaging arm 926, displacing the carrier 922 in a proximal direction. Accordingly, the ratchet slide 918 and carrier 922 will move proximally until the actuator 904 reaches the end of the stroke (e.g., either a partial stroke or a full stroke).

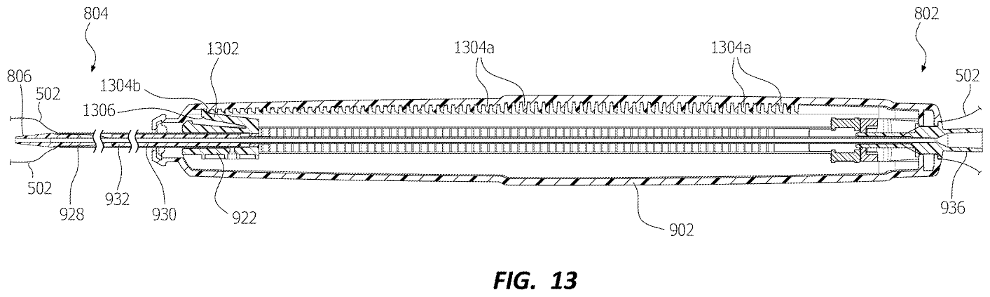

FIG. 13 is a cross-sectional view of the housing 902 and the carrier 922 in the same relative positions shown in FIG. 9. FIG. 13 further illustrates portions of the handle assembly 802, the delivery catheter assembly 804, and the guidewires 502 extending through the delivery catheter assembly 804 and the handle assembly 802. The cross-sectional plane of FIG. 13 extends along the longitudinal axis of the input portion 910. The cross-sectional plane of FIG. 13 extends horizontally, orthogonal to the cross-sectional planes of FIGS. 9, 10B, and 12.

As shown in FIG. 13, the carrier 922 includes a housing-engaging arm 1302 extending radially away from a longitudinal axis of the carrier 922. The housing 902 comprises a plurality of carrier-engaging housing lugs 1304a. A distal-most carrier-engaging housing lug 1304b may be denoted as reference numeral 1304b.

The housing-engaging arm 1302 may include an angled portion 1306 at a distal end of the housing-engaging arm 1302. The angled portion 1306 may extend radially away from the longitudinal axis of the carrier 922 at a greater angle than the housing-engaging arm 1302. In some examples, the angled portion 1306 can enhance engagement between the housing-engaging arm 1302 and a respective carrier-engaging housing lug 1304a. For example, as the carrier 922 moves in the proximal direction in response to the actuator 904 being depressed, the angled portion 1306 allows the housing-engaging arm 1302 to deflect radially as the angled portion 1306 contacts each of the respective carrier-engaging housing lug 1304a. When the actuator 904 is released, the carrier 922 may move in the distal direction, and the angled portion 1306 contacts a respective carrier-engaging housing lug 1304a to stop the distal movement of the carrier 922. The angled portion 1306 can provide clearance for the housing-engaging arm 1302, allowing the angled portion 1306 to engage a respective carrier-engaging housing lug 1304a (even when the carrier-engaging housing lugs 1304a are closely spaced) without adjacent carrier-engaging housing lugs 1304a interfering with the position of the housing-engaging arm 1302 and preventing full engagement.

Referring to FIGS. 9 through 13, as interaction between the actuator 904, the ratchet slide 918, and the carrier 922 displaces the carrier 922 with respect to the housing 902, the housing-engaging arm 1302 of the carrier 922 will deflect radially inward due to contact with respective ones of the carrier-engaging housing lugs 1304a. For example, as interaction between the distal-most carrier-engaging housing lug 1304b moves the carrier 922 proximally from the position shown in FIG. 13, the housing-engaging arm 1302 is displaced radially inward due to contact with the distal-most carrier-engaging housing lug 1304b. The housing-engaging arm 1302 moves radially inward until the housing-engaging arm 1302 clears the distal-most carrier-engaging housing lug 1304b, then moves radially outward to contact a portion of the housing 902 adjacent the carrier-engaging housing lugs 1304a. The point at which the housing-engaging arm 1302 moves proximal of the distal-most carrier-engaging housing lug 1304b may correspond to the stroke of the actuator 904 (e.g., a partial stroke or a full stroke), such that engagement between the housing-engaging arm 1302 and the next carrier-engaging housing lug 1304a (moving in a proximal direction) occurs at the end of the stroke. In some examples, each carrier-engaging housing lug 1304a (or at least a portion of each carrier-engaging housing lug 1304a) may be disposed such that a position of the carrier-engaging housing lug 1304a corresponds to a position of a carrier-engaging ratchet lug 924a.

A stroke of the actuator 904 can correspond to displacement of the carrier 922 past multiple carrier-engaging housing lugs 1304a. For example, for closely spaced carrier-engaging housing lugs 1304a, the actuator 904 may be configured to displace the carrier 922 over a semi-continuous range as the carrier 922 is advanced along the carrier-engaging housing lugs 1304a. Partially depressing the actuator 904 may displace the carrier 922 along and past a number of the carrier-engaging housing lugs 1304a, and upon release of the actuator 904, the carrier 922 may remain engaged with the most-recently passed carrier-engaging housing lug 1304a. Thus, increments of displacement of the carrier 922 may correspond to the spacing the carrier-engaging housing lugs 1304a, rather than the length of the stroke of the actuator 904.

As the actuator 904 is released following the stroke, interaction between the spring 916, the housing 902, and the actuator 904 will return the actuator 904 to the unconstrained position (e.g., the position shown in FIG. 9), as discussed above. Corresponding rotation of the actuator 904 about the pin aperture 908 may correlate to displacement of the ratchet slide-engaging portion 914, including a component of displacement in the distal direction. Interaction between the ratchet slide-engaging portion 914 and the actuator-engaging opening 1004 may correlate to distal displacement of the ratchet slide 918. Thus, when the actuator 904 is released at the end of a stroke, the actuator 904, the spring 916, and the ratchet slide 918 return to the same positions relative to the housing 902 illustrated in FIG. 9.

As the actuator 904 returns to the unconstrained position, interaction between the housing-engaging arm 1302 and a respective carrier-engaging housing lug 1304a prevents distal displacement of the carrier 922. Specifically, the distal surface of the angled portion 1306 of the housing-engaging arm 1302 will be in contact with a proximal-facing surface of the carrier-engaging housing lug 1304a. This interaction prevents the carrier 922 from returning to a pre-stroke position. In an example, the carrier 922 moves proximally such that the distal-most carrier-engaging housing lug 1304b displaces the housing-engaging arm 1302 inward during a stroke. Once the housing-engaging arm 1302 moves past the distal-most carrier-engaging housing lug 1304b the housing-engaging arm 1302 is displaced outward, and the housing-engaging arm 1302 may engage a proximal surface of the distal-most carrier-engaging housing lug 1304b following the stroke. Subsequent strokes move the carrier 922 along the plurality of carrier-engaging housing lugs 1304a in the proximal direction.

As the actuator 904 returns to the unconstrained position, radially inward displacement of the ratchet slide-engaging arm 926 of the carrier 922 allows the ratchet slide 918 to move distally with respect to the carrier 922. Engagement between the carrier 922 and the carrier-engaging housing lugs 1304a arrests the distal displacement of the carrier 922.

Further in FIGS. 9 through 13, with particular reference to the view of FIG. 12A, distal displacement of the ratchet slide 918 with respect to the carrier 922 creates interaction between the carrier-engaging ratchet lugs 924a and the angled portion 934 of the ratchet slide-engaging arm 926. This causes the ratchet slide-engaging arm 926 to displace radially inward. The distal-facing surfaces of the carrier-engaging ratchet lugs 924a may be angled to facilitate this interaction. During depression of the actuator 904, engagement between the distal-most carrier-engaging ratchet lug 924b can displace the carrier 922 in a proximal direction. During the return of the actuator 904, another carrier-engaging ratchet lug 924a (in a proximal direction) can cause the radially inward displacement of the ratchet slide-engaging arm 926 until the angled portion 934 of the ratchet slide-engaging arm 926 is proximal that carrier-engaging ratchet lug 924a. At that point, the ratchet slide-engaging arm 926 returns to a radially outward position (analogous to that shown in FIG. 12A), though the distal surface of the angled portion 934 of ratchet slide-engaging arm 926 is now engaged with a proximal face of another carrier-engaging ratchet lug 924a (in the proximal direction).

During a full stroke, engagement between a first carrier-engaging ratchet lug 924a can displace the carrier 922 in a proximal direction. During the return of the actuator 904, a plurality of the next carrier-engaging ratchet lugs 924a (in a proximal direction) can cause a plurality of radially inward displacements of the ratchet slide-engaging arm 926 as the angled portion 934 of the ratchet slide-engaging arm 926 moves proximally in relation to the carrier-engaging ratchet lugs 924a. Once the stroke is complete, the angled portion 934 of the ratchet slide-engaging arm 926 returns to a radially outward position (analogous to that shown in FIG. 12), with the distal surface of the angled portion 934 of ratchet slide-engaging arm 926 being engaged with a proximal face of a second carrier-engaging ratchet lug 924a (proximal to the first carrier-engaging ratchet lug 924a). A plurality of the carrier-engaging ratchet lugs 924a may be disposed between the first carrier-engaging ratchet lug 924a engaged during the stroke and the second carrier-engaging ratchet lug 924a engaged at the end of the stroke. For example, 1, 2, 3, 4, 5, 6, or more carrier-engaging ratchet lugs 924a may be disposed between the first carrier-engaging ratchet lug 924a engaged during a stroke and the second carrier-engaging ratchet lug 924a engaged at the end of the stroke.

Displacement of the ratchet slide 918 in response to a depression of the actuator 904 may correspond in magnitude to displacement of the ratchet slide 918 in response to a return of the actuator 904. As such, the ratchet slide 918 may return to an initial position after each depression and return of the actuator 904. One return of the actuator 904 following at least a partial stroke can move the ratchet slide 918 such that a plurality of carrier-engaging ratchet lugs 924a may serially engage the carrier 922 during the stroke.

Accordingly, as described above, depressing the actuator 904 for a full stroke, then allowing the actuator 904 to return to the unconstrained position, displaces the carrier 922 with respect to the housing 902 in discrete increments, corresponding to the distance between a plurality of carrier-engaging housing lugs 1304a along the longitudinal direction. Depressing the actuator 904 for a partial stroke, then allowing the actuator 904 to return to the unconstrained position, can displace the carrier 922 with respect to the 902 in discrete increments, corresponding to the distance between adjacent carrier-engaging housing lugs 1304a along the longitudinal direction.

As detailed below, the relative position of the carrier 922 with respect to the housing 902 may correlate to the degree of deployment of the fenestrated endovascular prosthesis 100 from the deployment device 800. Thus, visual, audible, and tactile feedback as to the position of the carrier 922 provides a user with information regarding deployment of the fenestrated endovascular prosthesis 100 during use of the deployment device 800. This information may correlate to increased control during deployment of the fenestrated endovascular prosthesis 100 as the practitioner quickly and intuitively can surmise the degree of deployment of the fenestrated endovascular prosthesis 100.

In some examples, at least a portion of the delivery catheter assembly 804 may lengthen and/or stretch during use of the deployment device 800. The configuration of the deployment device 800 (e.g., including semi-continuous carrier-engaging ratchet lugs 924a) allows for more than one increment of displacement of the carrier 922 in relation to the ratchet slide 918. The configuration of the deployment device 800 allows for finely tuned deployment of the fenestrated endovascular prosthesis 100. For example, the fenestrated endovascular prosthesis 100 can be deployed in increments of about 1 mm, about 2 mm, about 3 mm, about 4 mm, about 5 mm, or any other suitable increment.

The increments of displacement of the carrier 922 may be about 0.5 mm, about 1 mm, about 2 mm, about 3 mm, about 4 mm, about 5 mm, about 10 mm, about 25 mm, about 50 mm, about 100 mm, or any other suitable increment of displacement. The incremental displacement of the carrier 922 may facilitate partial deployment of the fenestrated endovascular prosthesis 100, allowing a practitioner to deploy the fenestrated endovascular prosthesis 100 in increments. This may allow the practitioner to adjust or confirm the position of the fenestrated endovascular prosthesis 100 between deployment increments.

FIG. 13 further illustrates guidewires 502 that extend through the delivery catheter assembly 804 and the handle assembly 802. As illustrated in FIG. 13, the guidewires 502 may enter the outer sheath 928 adjacent the distal tip 806. The guidewires 502 may extend through the outer sheath 928 and into the intermediate sheath 932. The guidewires 902 may extend out of the intermediate sheath 932 and into the handle assembly 802. The guidewires 902 may then extend through the handle assembly 802 and out of the handle assembly 802 adjacent the luer fitting 936. Guides may be formed within the deployment device 800 such that the guidewires 502 slidably extend through various components of the deployment device 800. For example, guides may be provided in the intermediate sheath 932, and the guidewires 502 may slidably extend through the guides in the intermediate sheath 932. This allows the guidewires 502 to be placed within the fenestrated endovascular prosthesis 100 when the fenestrated endovascular prosthesis 100 is positioned within the deployment device 800, and to move through the deployment device 800 as the deployment device 800 is removed from a patient's body. Positioning the guidewires 502 within the fenestrated endovascular prosthesis 100 prior to deployment within the patient's body aids in the deployment of secondary endovascular prostheses in fenestration tubes of the fenestrated endovascular prosthesis 100 after the fenestrated endovascular prosthesis 100 is deployed.

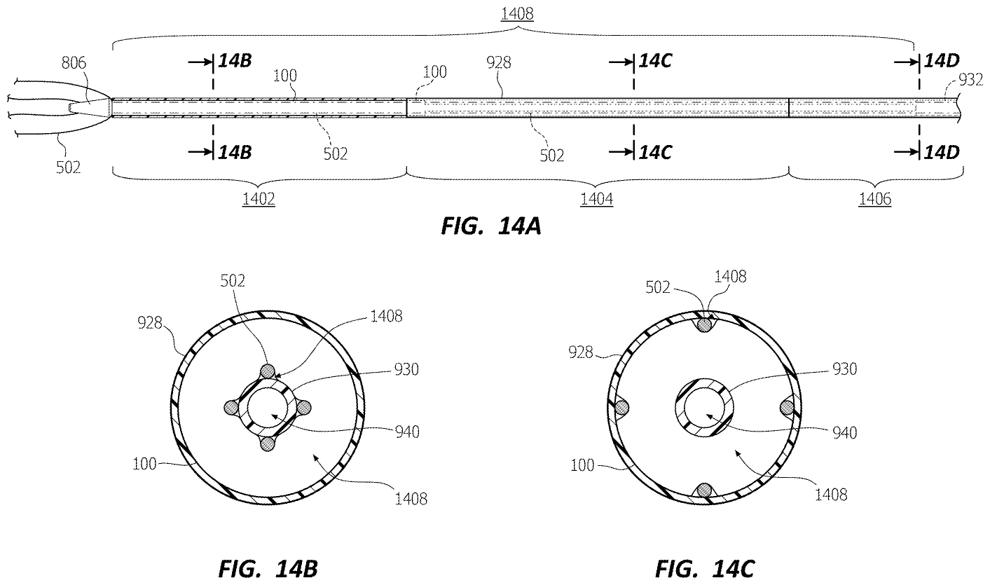

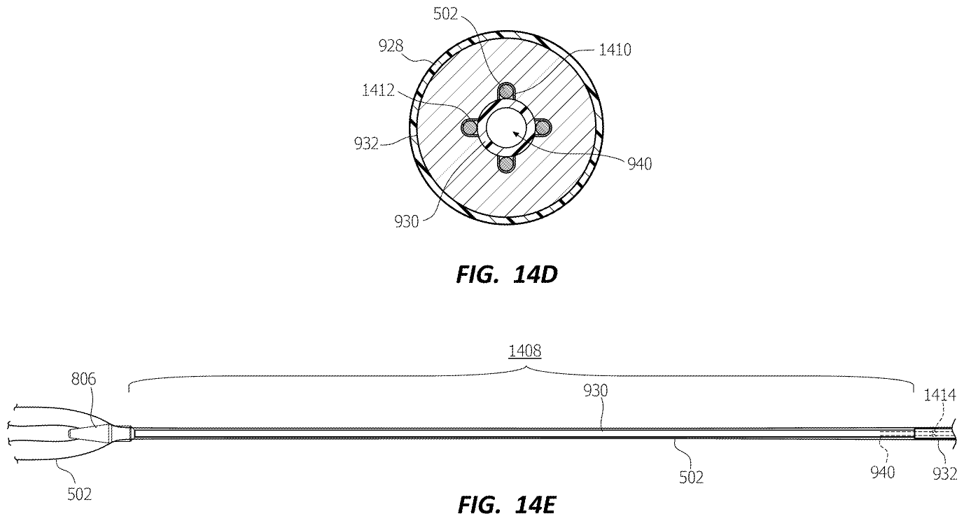

FIGS. 14A through 14E illustrate side views and cross-sectional views of portions of the delivery catheter assembly 804 of the deployment device 800. Specifically, FIG. 14A is a side view of a distal section of the delivery catheter assembly 804. FIG. 14B is a cross-sectional view of a portion of the delivery catheter assembly 804 of FIG. 14A along plane 14B-14B. FIG. 14C is a cross-sectional view of a portion of the delivery catheter assembly 804 of FIG. 14A along plane 14C-14C. FIG. 14D is a cross-sectional view of a portion of the delivery catheter assembly 804 of FIG. 14A along plane 14D-14D. FIG. 14E is a side view of the same longitudinal section of the delivery catheter assembly 804 illustrated in FIG. 14A, except that an outer sheath 928 and a fenestrated endovascular prosthesis 100 of FIG. 14A have been omitted in order to illustrate other components of the delivery catheter assembly 804.

Referring to FIGS. 8, 9, and 14A through 14E, the delivery catheter assembly 804 may be configured to deploy a fenestrated endovascular prosthesis 100 as the deployment device 800 is manipulated, as discussed above. The delivery catheter assembly 804 may comprise an outer sheath 928, extending from a handle assembly 802. The outer sheath 928 may be fixedly coupled to a carrier 922. The delivery catheter assembly 804 may further comprise an intermediate sheath 932 at least partially circumferentially surrounded by the outer sheath 928 and an inner sheath 930 at least partially surrounded by the outer sheath 928 and/or the intermediate sheath 932. The intermediate sheath 932 and the inner sheath 930 can be disposed within the outer sheath 928 and can be fixedly coupled to a housing 902. Proximal displacement of the carrier 922 with respect to the housing 902 will proximally displace the outer sheath 928 with respect to the intermediate sheath 932 and the inner sheath 930.

The outer sheath 928 may comprise a shaft section 1406 extending from the carrier 922 in a distal direction. At the distal end of the shaft section 1406 the outer sheath 928 may comprise a flex zone 1404 extending from the shaft section 1406 in the distal direction. The outer sheath 928 may comprise a pod 1402 extending from the flex zone 1404 in the distal direction. In the example illustrated in FIG. 14A, the pod 1402 of the outer sheath 928 is transparent.

The shaft section 1406 of the outer sheath 928 may have a different stiffness and/or durometer than the flex zone 1404 and/or the pod 1402. The flexibility toward the distal end of the outer sheath 928 may improve trackability of the delivery catheter assembly 804 over a guidewire (e.g., a deployment guidewire) and may be less traumatic when the delivery catheter assembly 804 is inserted into a patient. Providing the shaft section 1406 that is relatively stiff may result in the delivery catheter assembly 804 being more kink resistant, as well as being better at transmitting displacement and/or torque along the shaft section 1406.

The outer sheath 928 may be configured to retain the fenestrated endovascular prosthesis 100 in a crimped or otherwise constrained state. Removal of the outer sheath 928 from around the fenestrated endovascular prosthesis 100 may allow the fenestrated endovascular prosthesis 100 to self-expand, and thereby deploy at a treatment site. The pod 1402, the flex zone 1404, and the shaft section 1406 may be any suitable length, and may be any length relative to one another. In some examples, the fenestrated endovascular prosthesis 100 may be provided in one, two, or all three sections of the outer sheath 928 (e.g., the fenestrated endovascular prosthesis 100 may be provided in the pod 1402, the flex zone 1404, and/or the shaft section 1406). For example, in the illustrated example, an annular space 1408 is configured to receive the fenestrated endovascular prosthesis 100 and extends in the pod 1402, the flex zone 1404, and the shaft section 1406. In some examples, the annular space 1408 may extend in only the pod 1402, in the pod 1402 and the flex zone 1404, or the like. In other words, the delivery catheter assembly 804 may be configured to retain the fenestrated endovascular prosthesis 100 in the pod 1402; in the pod 1402 and the flex zone 1404; in the pod 1402, the flex zone 1404, and the shaft section 1406; or the like.

The distal tip 806 of the delivery catheter assembly 804 may be coupled to and/or integrally formed with the inner sheath 930. The inner sheath 930 may be fixed to the housing 902. For example, the proximal end of the inner sheath 930 may be fixed to the housing 902. The intermediate sheath 932 may also be fixed to the housing 902, and may extend over a portion of the inner sheath 930. The intermediate sheath 932 and inner sheath 930 may or may not be directly fixed to each other. In some examples, the intermediate sheath 932 may be a close slip fit over the inner sheath 930.

The inner sheath 930 extends distally beyond a distal end of the intermediate sheath 932. This creates the annular space 1408 between the inner sheath 930 and the outer sheath 928 adjacent the distal tip 806. The annular space 1408 extends proximally from the distal tip 806 to the distal end of the intermediate sheath 932. In examples in which the intermediate sheath 932 is omitted, the annular space 1408 may extend to the housing 902. The annular space 1408 may be configured to retain the fenestrated endovascular prosthesis 100.

As the carrier 922 is manipulated to incrementally displace the carrier 922 in the proximal direction relative to the housing 902, the outer sheath 928 is incrementally displaced in the proximal direction relative to the inner sheath 930 and the intermediate sheath 932. The distal end of the intermediate sheath 932 interacts with the proximal end of the fenestrated endovascular prosthesis 100, preventing the fenestrated endovascular prosthesis 100 from being displaced with the outer sheath 928. Thus, the fenestrated endovascular prosthesis 100 is incrementally exposed. As the fenestrated endovascular prosthesis 100 is exposed, the fenestrated endovascular prosthesis 100 self-expands and deploys.

As illustrated in FIGS. 14A and 14B, the guidewires 502 may extend from outside the delivery catheter assembly 804 into a space inside the outer sheath 928 adjacent the distal tip 806. In some examples, grooves may be formed in the distal tip 806, and the guidewires 502 may extend in the grooves and into the outer sheath 928. The guidewires 502 may extend into an inner portion of the fenestrated endovascular prosthesis 100 at a proximal end of the outer sheath 928. For example, the guidewires 502 may extend into a bore 106 of the fenestrated endovascular prosthesis 100. The guidewires 502 may extend along outer surfaces of the inner sheath 930 in the cross-sectional view of FIG. 14B, which may be in a proximal portion 112 of the fenestrated endovascular prosthesis 100. The guidewires 502 may extend through fenestration tubes 104 of the fenestrated endovascular prosthesis 100 such that the guidewires 502 extend from inside the fenestrated endovascular prosthesis 100 to outside the fenestrated endovascular prosthesis 100. As illustrated in FIGS. 14A and 14C, the guidewires 502 may extend inside the outer sheath 928 along outer surfaces of the fenestrated endovascular prosthesis 100, which may be in a distal portion 114 of the fenestrated endovascular prosthesis 100. The guidewires 502 may extend along inner surfaces of the outer sheath 928 in the cross-sectional view of FIG. 14C. FIG. 14A illustrates the guidewires 502 as moving from inside the fenestrated endovascular prosthesis 100 to outside the fenestrated endovascular prosthesis 100 in the flex zone 1404; however, the guidewires 502 may move from inside the fenestrated endovascular prosthesis 100 to outside the fenestrated endovascular prosthesis 100 anywhere along the length of the annular space 1408.

As illustrated in FIGS. 14D and 14E, the guidewires 502 may extend from the annular space 1408 into the intermediate sheath 932. In some examples, one or more guide lumens 1412 may extend longitudinally through a wall of the intermediate sheath 932. Each of the guide lumens 1410 can be configured to slidingly receive a respective guidewire 502. Specifically, the guide lumens 1410 can be configured to allow the guidewires 502 to slide through the delivery device 800 when the guidewires 502 and the fenestrated endovascular prosthesis 100 are loaded into the delivery device 800, and to allow the delivery device 800 to slide along the guidewires 502 as the delivery device 800 is removed from a patient's body. The guide lumens 1410 may be configured to prevent tangling and/or crossing of the guidewires 502 as the guidewires 502 extend through the fenestrated endovascular prosthesis 100, the delivery catheter assembly 804, and the handle assembly 802, as illustrated in FIGS. 8, 9, 14A, and 14E. In some examples, the guide lumens 1410 may include blood flow restrictor members 1412 configured to prevent or restrict blood flow through the guide lumens 1410 and into the handle assembly 802. The blood flow restrictor members 1412 can be septums, membranes, or the like, and can be disposed over or within the guide lumens 1410. The blood flow restrictor members 1412 can be configured to be pierced by and to seal around the guidewires 502. Other forms of the blood flow restrictor members 1412 are within the scope of this disclosure. The guide lumens 1410 may be provided along inner surfaces of the intermediate sheath 932 and outer surfaces of the inner sheath 930.

In some examples, one or more fluid apertures 1414 extending through the wall of the intermediate sheath 932 and the wall of the inner sheath 930 may be included. The fluid apertures 1414 may be in fluid communication with the lumen 940. The fluid apertures 1414 may provide fluid communication between the annular space 1408 and the lumen 940 such that fluid within the lumen 940 can move through the fluid apertures 1414 and into the annular space 1408. This communication may be used to flush the annular space 1408 during use. In some examples, the fluid apertures 1414 may be configured to remove air or other unwanted materials from the annular space 1408 or from around the fenestrated endovascular prosthesis 100.

Various components of the delivery catheter assembly 804 may be specially configured to interface with the guidewires 502. For example, the distal tip 806 may include the grooves through which the guidewire 502 extend, openings may be provided in the outer sheath 928, and the guide lumens 1410 may be provided in the intermediate sheath 932. This aids in the deployment device 800 being able to slide along the guidewires 502 as the guidewires 502 and the fenestrated endovascular prosthesis 100 are loaded into the deployment device 800, and as the deployment device 800 is removed from a patient's body after the fenestrated endovascular prosthesis 100 is deployed at a treatment site. After the fenestrated endovascular prosthesis 100 is deployed, the guidewires 502 can be used to deploy secondary endovascular prostheses.

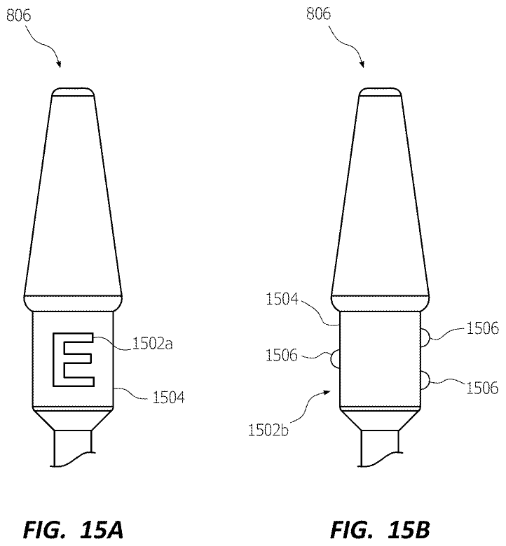

As depicted in FIGS. 15A and 15B, the distal tip 806 may include various orientation indicia configured to provide radiographic feedback to a user of the deployment device 800 regarding the rotational orientation of the delivery catheter assembly 804 relative to the treatment site. This may be used to properly position the delivery catheter assembly 804 and the fenestrated endovascular prosthesis 100 relative to branch arteries and the like at the treatment site. In some examples, the orientation of the handle assembly 802 of the deployment device 800 relative to a patient's body may be used to determine the orientation of the delivery catheter assembly 804 and the fenestrated endovascular prosthesis 100 relative to the treatment site in addition to or in place of the orientation indicia discussed with respect to FIGS. 15A and 15B.

FIG. 15A illustrates an orientation indicium 1502a included in the distal tip 806. A shape may be cut in the band. The shape may include a longitudinal segment and two or more segments, for example three segments, extending laterally from the longitudinal segment. For example, in the example of FIG. 15A, the shape is a capital “E.” In use, under radiographic imaging, the user can observe the direction that the laterally extending segments point (e.g., left or right) in order to determine the rotational orientation (e.g., anteriorly or posteriorly with respect to the patient's body) of the orientation indicium 1502a. For example, when the laterally extending segments point to the left, the orientation indicium 1502a is oriented posteriorly and when the laterally extending segments point to the right, the orientation indicium 1502a is oriented anteriorly. The rotational orientation of the orientation indicium 1502a can relate to a rotational orientation of the fenestrated endovascular prosthesis 100 and can help to provide proper rotational orientation of the fenestrated endovascular prosthesis 100 with respect to the branch arteries or the like at the treatment site. Other forms of orientation indicia are within the scope of this disclosure.

FIG. 15B illustrates another example of an orientation indicium 1502b. As depicted, an orientation indicium 1502b may include a radiopaque marker band 1504 coupled to the distal tip 806. Bumps 1506 may be disposed on the radiopaque marker band 1504. Two or more bumps 1506 may be disposed adjacent each other on one side of the radiopaque marker band 1504 and a single bump 1506 may be disposed circumferentially opposite the two or more bumps 1506. In use, under radiographic imaging the user can observe the side the single bump 1506 is on (e.g., left or right) in order to determine the rotational orientation (e.g., anteriorly or posteriorly with respect to the patient's body) of the orientation indicium 1502b. For example, when the single bump 1506 is on the left side, the orientation indicium 1502b is oriented posteriorly and when the single bump 1506 is on the right side, the orientation indicium 1502b is oriented anteriorly. The rotational orientation of the orientation indicium 1502b can relate to a rotational orientation of the fenestrated endovascular prosthesis 100 and can help to provide proper rotational orientation of the fenestrated endovascular prosthesis 100 with respect to the branch arteries or the like at the treatment site.

FIGS. 16A and 16B illustrate examples of fenestration dilators in accordance with some examples. The fenestration dilators may be configured to transition fenestration tubes 104 of a fenestrated endovascular prosthesis 100 from a sealed configuration to an open configuration, allowing a secondary endovascular prosthesis to be deployed in the un-sealed fenestration tubes 104.

In FIG. 16A, a fenestration dilator 1600a includes an elongate tubular body 1602a and a coupling 1604a coupled to a proximal end of the tubular body 1602a. The tubular body 1602a may include a shaft portion 1606a and a tip portion 1608a. In the example illustrated in FIG. 16A, the shaft portion 1606a is a substantially straight cylinder. The shaft portion 1606a may have an outer diameter substantially equivalent to a diameter of a lumen 206 of a respective fenestration tube 104. In some examples, the outer diameter of the shaft portion 1606a can be sized relative to an inner diameter of a lumen 206 of a respective fenestration tube 104 in which the fenestration dilator 1600a is to be inserted. For example, a ratio of an outer diameter of the shaft portion 1606a to an inner diameter of a lumen 206 of a respective fenestration tube 104 in which the fenestration dilator 1600a is to be inserted can be in a range from about 0.50 to about 0.95. This provides that the fenestration dilator 1600a is sufficiently narrow to pass through the lumen 206 of the fenestration tube 104, and the fenestration dilator 1600a is sufficiently broad to have the column strength to push through a seal region 216 of the fenestration tube 104. The tip portion 1608a can taper distally such that a distal end of the tip portion 1608a closely fits around a guidewire 502 that extends through the fenestration tube 104. This allows for the tip portion 1608a to pass into the lumen 206 of the fenestration tube 104 and through a seal region 216 of the respective fenestration tube 104 with minimal resistance when the fenestration tube 104 is transitioned from the sealed configuration to the open configuration. The coupling 1604a can be in fluid communication with a bore extending through the tubular body 1602a, and may couple the tubular body 1602a to a deployment device. The deployment device may be a secondary deployment device, which may be similar to the deployment device 800.

In FIG. 16B, a fenestration dilator 1600b includes an elongate tubular body 1602b and a coupling 1604b coupled to a proximal end of the tubular body 1602b. The tubular body 1602b may include a shaft portion 1606b and a tip portion 1608b. The shaft portion 1606b may include a proximal section 1610, an intermediate section 1612, and a distal section 1614. The intermediate section 1612 may be disposed between the proximal section 1610 and the distal section 1614. A transition section 1616a may be disposed between the proximal section 1610 and the intermediate section 1612 and a transition section 1616b may be disposed between the intermediate section 1612 and the distal section 1614. A first outer diameter of the proximal section 1610 can be larger than a second outer diameter of the intermediate section 1612, and the second outer diameter of the intermediate section 1612 can be larger than a third outer diameter of the distal section 1614. The first outer diameter of the proximal section 1610 may be substantially equivalent to a diameter of a lumen 206 of a respective fenestration tube 104. In some examples, the first outer diameter, the second outer diameter, and the third outer diameter of the shaft portion 1606b can be sized relative to an inner diameter of a lumen 206 of a respective fenestration tube 104 in which the fenestration dilator 1600b is to be inserted. For example, ratios of the first outer diameter, the second outer diameter, and the third outer diameter of the shaft portion 1606b to an inner diameter of a lumen 206 of a respective fenestration tube 104 in which the fenestration dilator 1600b is to be inserted can be in a range from about 0.50 to about 0.95. This provides that the fenestration dilator 1600b is sufficiently narrow to pass through the lumen 206 of the fenestration tube 104, and the fenestration dilator 1600a is sufficiently broad to have the column strength to push through a seal region 216 of the fenestration tube 104. The variations in diameters along the length of the shaft portion 1606b can effect variations in the opening of the fenestration tubes 104. In some examples, the variations in diameters along the length of the shaft portion 1606b can be provided such that the fenestration dilator 1600b can be used to open fenestration tubes 104 having different diameters.

In some examples, the proximal section 1610, the intermediate section 1612, and the distal section 1614 can be configured to open a respective fenestration tube 104 to one or more of the outer diameters. In some examples, each of the proximal section 1610, the intermediate section 1612, and the distal section 1614 can be configured to open a lumen 206 of a respective fenestration tube 104 having a different diameter. In other words, the proximal section 1610 can be configured to open a lumen 206 of a fenestration tube 104 having the first diameter; the intermediate section 1612 can be configured to open a lumen 206 of a fenestration tube 104 having the second diameter; and the distal section 1614 can be configured to open a lumen 206 of a fenestration tube 104 having the third diameter. The tip portion 1608b can taper distally such that a distal end of the tip portion 1608b closely fits around a guidewire 502 that extends through the fenestration tube 104. This allows for the tip portion 1608a to pass through a seal region 216 of the respective fenestration tube 104 with minimal resistance when the fenestration tube 104 is transitioned from the sealed configuration to the open configuration. The coupling 1604b can be in fluid communication with a bore extending through the tubular body 1602b, and may couple the tubular body 1602b to a deployment device. The deployment device may be a secondary deployment device, which may be similar to the delivery deployment device 800.

FIGS. 17A and 17B illustrate examples of a fenestration balloon dilators in accordance with some examples. The fenestration balloon dilators may be configured to transition the fenestration tubes 104 of a fenestrated endovascular prosthesis 100 from a sealed configuration to an open configuration, allowing a secondary endovascular prosthesis to be deployed in any of the un-sealed fenestration tubes 104.

In FIG. 17A, a fenestration balloon dilator 1700a can include an elongate tubular body 1702a and a coupling 1704a coupled to a proximal end of the tubular body 1702a. The tubular body 1702a may include a shaft portion 1708a and a tip portion 1706a. The shaft portion 1708a may include an expandable member 1710a (also referred to as a balloon). The expandable member 1710a may have an outer diameter substantially equivalent to a diameter of a lumen 206 of a respective fenestration tube 104 when the expandable member 1710a is expanded. In some examples, an outer diameter of the shaft portion 1708a (e.g., the outer diameter of the expandable member 1710a when the expandable member 1710a is expanded) can be sized relative to an inner diameter of a lumen 206 of a respective fenestration tube 104 in which the fenestration balloon dilator 1700a is to be inserted. For example, a ratio of an outer diameter of the shaft portion 1708a to an inner diameter of a lumen 206 of a respective fenestration tube 104 in which the fenestration balloon dilator 1700a is to be inserted can be in a range from about 0.50 to about 0.95. This provides that the fenestration balloon dilator 1700a is sufficiently narrow to pass through the lumen 206 of the fenestration tube 104, and the fenestration balloon dilator 1700a is sufficiently broad to have the column strength to push through a seal region 216 of the fenestration tube 104.

The tip portion 1706a can taper distally such that a distal end of the tip portion 1706a closely fits around a guidewire 502 that extends through the fenestration tube 104. This allows for the tip portion 1706a to pass into the lumen 206 of the fenestration tube 104 and through a seal region 216 of the respective fenestration tube 104 with minimal resistance when the fenestration tube 104 is transitioned from the sealed configuration to the open configuration. The coupling 1704a may include a primary port 1712 in fluid communication with a bore extending through the tubular body 1702a and an expansion port 1714 (also referred to as a balloon port) in fluid communication with the expandable member 1710a. The coupling 1704a may couple the tubular body 1702a to a deployment device. The deployment device may be a secondary deployment device, which may be similar to the delivery deployment device 800.

In FIG. 17B, a fenestration balloon dilator 1700b can include an elongate tubular body 1702b and a coupling 1704b coupled to a proximal end of the tubular body 1702bb. The tubular body 1702b may include a shaft portion 1708b and a tip portion 1706b. The shaft portion 1708b may include an expandable member 1710b (also referred to as a balloon), which may include a proximal section 1716, an intermediate section 1718, and a distal section 1720. The intermediate section 1718 may be disposed between the proximal section 1716 and the distal section 1720. A transition section 1722a may be disposed between the proximal section 1716 and the intermediate section 1718, and a transition section 1722b may be disposed between the intermediate section 1718 and the distal section 1720. When the expandable member 1710b is in an expanded state, a first outer diameter of the proximal section 1716 can be larger than a second outer diameter of the intermediate section 1718, and the second outer diameter of the intermediate section 1718 can be larger than a third outer diameter of the distal section 1720. The first outer diameter of the proximal section 1716 may be substantially equivalent to a diameter of a lumen 206 of a respective fenestration tube 104. In some examples, the first outer diameter, the second outer diameter, and the third outer diameter of the shaft portion 1708b can be sized relative to an inner diameter of a lumen 206 of a respective fenestration tube 104 in which the fenestration balloon dilator 1700b is to be inserted. For example, ratios of the first outer diameter, the second outer diameter, and the third outer diameter of the shaft portion 1708b to an inner diameter of a lumen 206 of a respective fenestration tube 104 in which the fenestration balloon dilator 1700b is to be inserted can be in a range from about 0.50 to about 0.95. This provides that the fenestration balloon dilator 1700b is sufficiently narrow to pass through the lumen 206 of the fenestration tube 104, and the fenestration balloon dilator 1700b is sufficiently broad to have the column strength to push through a seal region 216 of the fenestration tube 104. The variations in diameters along the length of the shaft portion 1708b can effect variations in the opening of the fenestration tubes 104. In some examples, the variations in diameters along the length of the shaft portion 1708b can be provided such that the fenestration balloon dilator 1700b can be used to open fenestration tubes 104 having different diameters.

In some examples, the proximal section 1716, the intermediate section 1718, and the distal section 1720 can be configured to open a respective fenestration tube 104 to one or more of the outer diameters. In some examples, each of the proximal section 1716, the intermediate section 1718, and the distal section 1720 can be configured to open a lumen 206 of a respective fenestration tube 104 having a different diameter. In other words, the proximal section 1716 can be configured to open a lumen 206 of a fenestration tube 104 having the first diameter; the intermediate section 1718 can be configured to open a lumen 206 of a fenestration tube 104 having the second diameter; and the distal section 1720 can be configured to open a lumen 206 of a fenestration tube 104 having the third diameter.

The tip portion 1706b can taper distally such that a distal end of the tip portion 1706b closely fits around a guidewire 502 that extends through the fenestration tube 104. This allows for the tip portion 1706b to pass through a seal region 216 of the respective fenestration tube 104 with minimal resistance when the fenestration tube 104 is transitioned from the sealed configuration to the open configuration. The coupling 1704b may include a primary port 1712 in fluid communication with a bore extending through the tubular body 1702b and an expansion port 1714 (also referred to as a balloon port) in fluid communication with the expandable member 1710b. The coupling 1704b may couple the tubular body 1702b to a deployment device. The deployment device may be a secondary deployment device, which may be similar to the delivery deployment device 800.

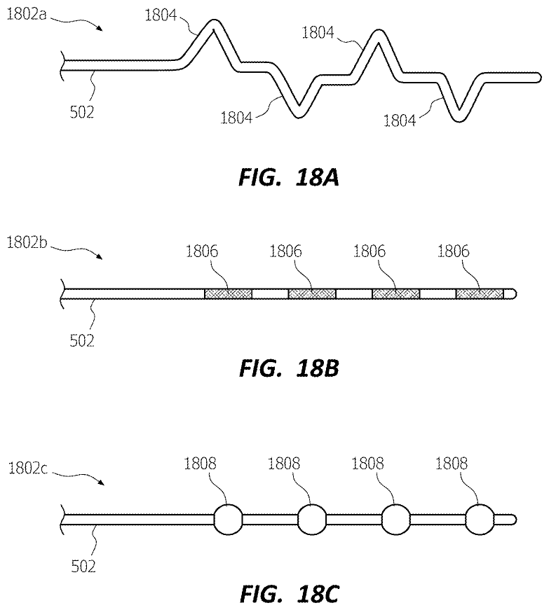

FIGS. 18A through 18C illustrate examples of identification features that may be included at end portions of the guidewires 502 in accordance with some examples. Matching identification features may be disposed at each end of each of the guidewires 502. The identification features can be configured to allow a user of a deployment device 800 used to deploy a fenestrated endovascular prosthesis 100 to identify both ends of each respective guidewire 502 extending through the fenestrated endovascular prosthesis 100 (e.g., extending through a specific fenestration tube 104 of the fenestrated endovascular prosthesis 100). This may be particularly useful for examples in which two or more of the guidewires 502 extend through an implanted fenestrated endovascular prosthesis 100.

As an example, a plurality of the guidewires 502 may be positioned in a patient. The guidewires 502 may extend into a first vascular access site (e.g., a femoral or a jugular access site), through a vascular treatment site, and out of a second vascular access site (e.g., the other of the femoral or the jugular access site). A deployment device 800, including a fenestrated endovascular prosthesis 100, may be advanced along the guidewires 502, and the fenestrated endovascular prosthesis 100 may be deployed at the vascular treatment site. The deployment device 800 may then be removed from the patient's body. In some examples, orientation indicia (such as the orientation indicia discussed above with respect to FIGS. 15A and 15B) may be used to determine an orientation of the fenestrated endovascular prosthesis 100 in the patient's body. Based on the orientation of the fenestrated endovascular prosthesis 100 and a configuration of branched arteries or the like within the patient's body, fenestration tubes 104 of the fenestrated endovascular prosthesis 100 in which secondary prostheses are to be deployed may be selected. The fenestration tubes 104 may be selected based on their proximity to the branched arteries to which secondary endovascular prostheses are to be deployed, based on diameters or lengths of the fenestration tubes 104, or the like.

Fenestration dilators (such as the fenestration dilators discussed above with respect to FIGS. 16A through 17B) may be advanced along selected guidewires 502 to open the selected fenestration tubes 104, and secondary prostheses may then be advanced along the selected guidewires 502 and deployed in the selected fenestration tubes 104. In some examples, selected guidewires 502 may be replaced by secondary guidewires that extend from a vascular access into the branched arteries, and the secondary prostheses may be advanced along the secondary guidewires. The identification features help the practitioner to identify each of the guidewires 502 at both the first vascular access site and the second vascular access site such that the fenestration dilators, secondary guidewires, secondary prostheses, and/or other treatments are advanced along desired guidewires 502 and may be deployed in selected fenestration tubes 104.

In FIG. 18A, an identification feature 1802a can include one or more bends or kinks 1804, provided in a guidewire 502. FIG. 18A illustrates the guidewire 502 including four kinks 1804 for the identification feature 1802a. In an example in which four guidewires 502 are provided and extend through four fenestration tubes 104 of a fenestrated endovascular prosthesis 100, a first guidewire 502 may include one kink 1804 in an identification feature 1802a; a second guidewire 502 may include two kinks 1804 in an identification feature 1802a; a third guidewire 502 may include three kinks 1804 in an identification feature 1802a; and a fourth guidewire 502 (illustrated in FIG. 18A) may include four kinks 1804 in an identification feature 1802a. Although the identification feature 1802a of the guidewire 502 illustrated in FIG. 18A is illustrated as including four kinks 1804 that extend in opposite directions, any number of kinks 1804, such as a greater number or fewer of the kinks 1804 may be provided. Moreover, adjacent kinks 1804 may extend in the same or opposite directions, and may be formed in any desired shape or configuration. For example, the kinks 1804 may be V-shaped, U-shaped, or the like. The number of kinks 1804, shapes of the kinks 1804, and the like may be used as the identification feature 1802a to identify specific guidewires 502.

The kinks 1804 may be offset from each other along the length of the guidewires 502. In some embodiments each kink 1804 may form an angle of between 5° and 90°, including from 10° to 20°. The angles formed by the kinks 1804 may be configured to readily distinguish the pattern formed on one wire from that of another. Thus, the kinks 1804 may be configured with sufficient size and/or spacing to be visually identifiable by a user of the guidewires 502, while also allowing the guidewires 502 to be smoothly advanced through a patient's body, through the fenestration tubes 104 of a fenestrated endovascular prosthesis 100, through a deployment device 800, through a secondary deployment device, and the like. Specifically, providing the kinks 1804 that are bent at too great of an angle relative to the longitudinal axis of the guidewires 502 may cause the guidewires 502 to get stuck in any of the patient's body, the fenestration tubes 104 of a fenestrated endovascular prosthesis 100, the deployment device 800, the secondary deployment device, or the like.

In FIG. 18B, an identification feature 1802b can include one or more marker bands 1806, provided in a guidewire 502. The marker bands 1806 may be printed, etched, or the like on surfaces of respective guidewires 502. FIG. 18B illustrates the guidewire 502 including four marker bands 1806 for the identification feature 1802b. In an example in which four guidewires 502 are provided and extend through four fenestration tubes 104 of a fenestrated endovascular prosthesis 100, a first guidewire 502 may include one marker band 1806 in an identification feature 1802b; a second guidewire 502 may include two marker bands 1806 in an identification feature 1802b; a third guidewire 502 may include three marker bands 1806 in an identification feature 1802b; and a fourth guidewire 502 (illustrated in FIG. 18B) may include four marker bands 1806 in an identification feature 1802b. Although the identification feature 1802b of the guidewire 502 illustrated in FIG. 18B is illustrated as including four marker bands 1806, any number of marker bands 1806, such as a greater number or fewer of the marker bands 1806 may be provided. Moreover, the marker bands 1806 may be the same color as one another, or different colors from one another. As such, the number of marker bands 1806, the color of the marker bands 1806, and the like may be used as the identification features 1802b to identify specific guidewires 502. The guidewires 502 may be relatively thin, and the marker bands 1806 may be relatively small. As such, the marker bands 1806 can be formed of materials that can be visually enhanced, such as through reflectivity, through the use of black light, or the like.

In FIG. 18C, an identification feature 1802c can include one or more marker nubs 1808, provided in a guidewire 502. The marker nubs 1808 may comprise the same material as the guidewires 502, or may be a different material formed around the guidewires 502. FIG. 18C illustrates the guidewire 502 including four marker nubs 1808 for the identification feature 1802c. In an example in which four guidewires 502 are provided and extend through four fenestration tubes 104 of a fenestrated endovascular prosthesis 100, a first guidewire 502 may include one marker nub 1808 in an identification feature 1802c; a second guidewire 502 may include two marker nubs 1808 in an identification feature 1802c; a third guidewire 502 may include three marker nubs 1808 in an identification feature 1802c; and a fourth guidewire 502 (illustrated in FIG. 18B) may include four marker nubs 1808 in an identification feature 1802c. Although the identification feature 1802c of the guidewire 502 illustrated in FIG. 18C is illustrated as including four marker nubs 1808, any number of marker nubs 1808, such as a greater number or fewer of the marker nubs 1808 may be provided. Moreover, the marker nubs 1808 may be the same shape, size, and/or color as one another, or different shapes, sizes, and/or colors from one another. For example, although the marker nubs 1808 are illustrated as being spherical, the marker nubs 1808 may be conical, or may have any other suitable shape. The marker numbs 1808 may be referred to as marker bumps, marker balls, or the like. As such, the number of marker nubs 1808, the color of the marker nubs 1808, the shape of the marker nubs 1808, the sizes of the marker nubs 1808, and the like may be used as the identification features identification features 1802c to identify specific guidewires 502.

Ratios of diameters of respective ones of the marker nubs 1808 to a diameter of the guidewire 502 on which the marker nubs 1808 are disposed may be in a range from about 1.02 to about 1.25. This provides the marker nubs 1808 with sufficient size to be recognized by a user of the guidewires 502, while allowing the guidewires 502 to be smoothly advanced through a patient's body, through the fenestration tubes 104 of a fenestrated endovascular prosthesis 100, through a deployment device 800, through a secondary deployment device, and the like. Specifically, providing the marker nubs 1808 with too great of diameters may cause the guidewires 502 to get stuck in any of the patient's body, the fenestration tubes 104 of a fenestrated endovascular prosthesis 100, the deployment device 800, the secondary deployment device, or the like.

Although three guidewire identification features have been discussed with respect to FIGS. 18A through 18C, additional guidewire identification features may be provided in addition to or in place of the described guidewire identification features. For example, the guidewires 502 may be provided with different lengths to identify particular guidewires 502. In some examples, the guidewires 502 may include features, such as kinks, marker bands, nubs, or the like, which are disposed along the length of the guidewires 502 rather than at end portions of the guidewires 502, and which are detectable through fluoroscopy or the like.

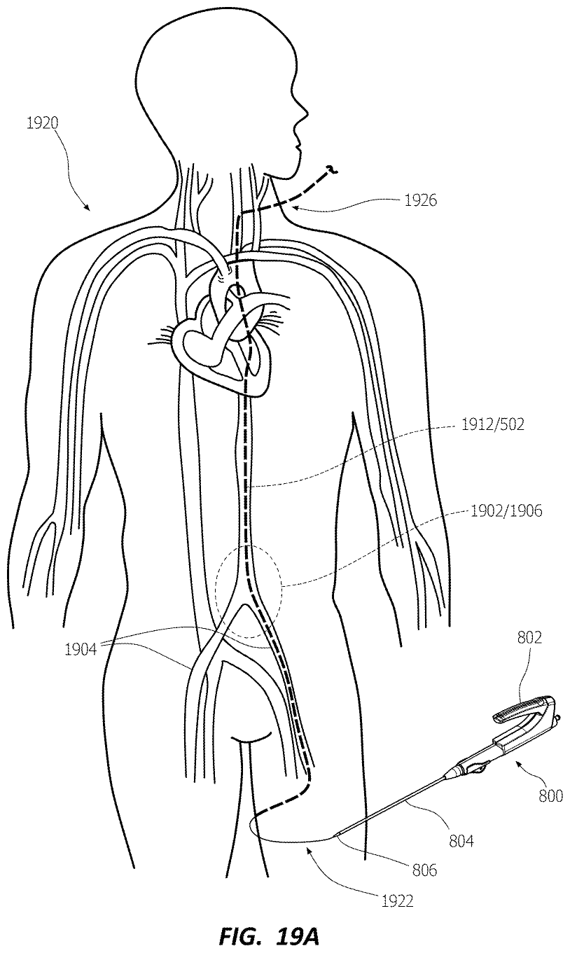

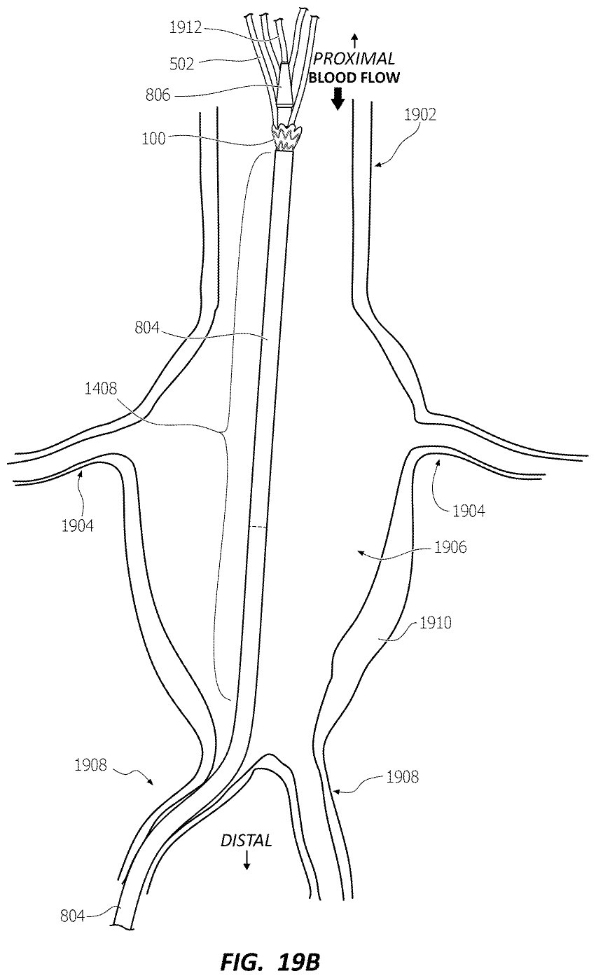

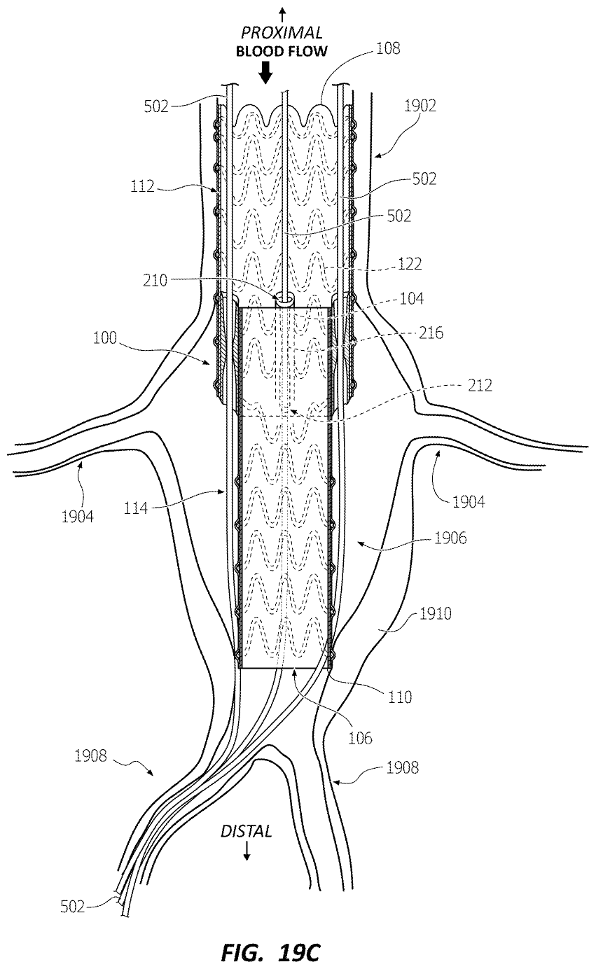

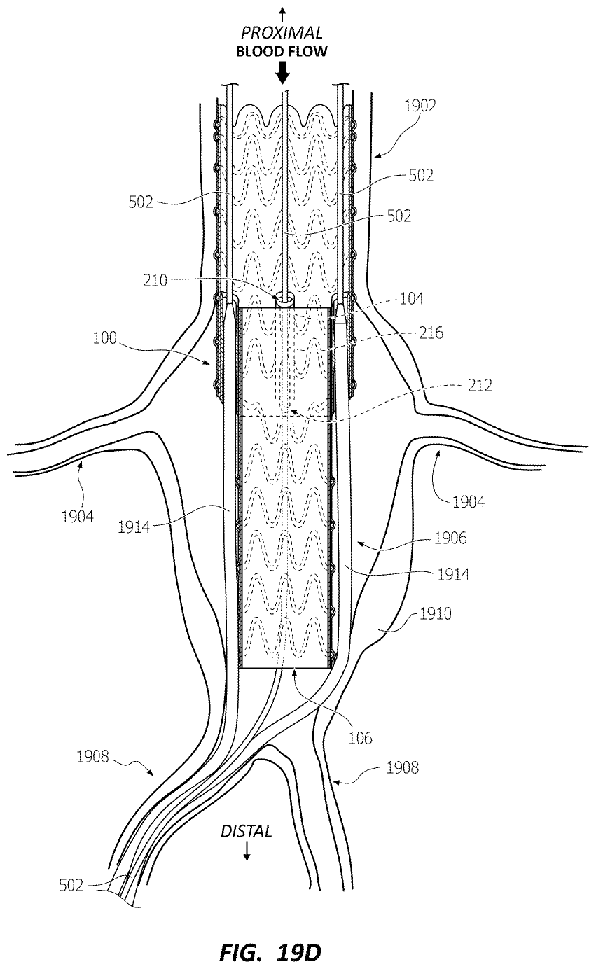

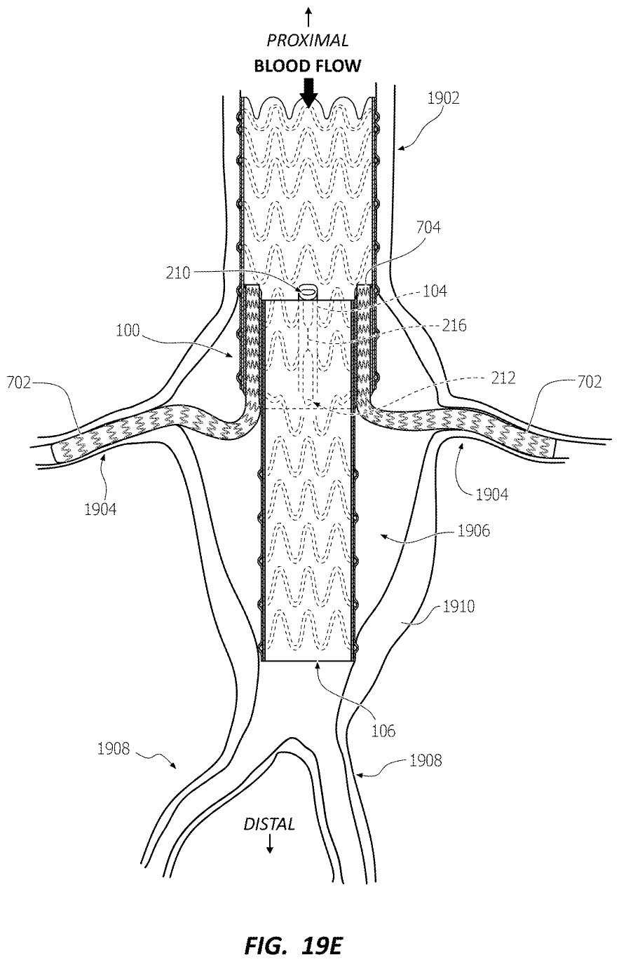

FIGS. 19A through 19E illustrate a method of implanting a fenestrated endovascular prosthesis 100 and secondary endovascular prostheses 702 (illustrated in FIG. 19E) in a diseased blood vessel 1902 including branch vessels 1904. The fenestrated endovascular prosthesis 100 may be deployed using a deployment device 800 in any diseased arterial or venous vessel of a patient 1920, including in arteries or vessels having branches, such as an aortic arch, a thoracic aorta, an abdominal aorta, an inferior vena cava, or the like. Although the fenestrated endovascular prosthesis 100 is illustrated as being deployed in a diseased blood vessel 1902 including various branched arteries, the fenestrated endovascular prosthesis 100 can be deployed in portions of vessels or arteries that do not include branched arteries. The diseased blood vessel 1902 may include a diseased section 1906, from which the branch vessels 1904 branch off. The diseased section 1906 may be an aneurysm, an aortic dissection, or any other type of vascular disease. In some examples, the branch vessels 1904 may include renal arteries, a brachiocephalic trunk, a left common carotid artery, a left subclavian artery, bronchial arteries, esophageal arteries, intercostal arteries, mediastinal arteries, pericardial arteries, mesenteric arteries, gonadal arteries, lumbar arteries, or the like. The branch vessels 1904 may extend radially outward from the diseased section 1906 of the diseased blood vessel 1902. In some examples, the diseased section 1906 may be adjacent distal branch vessels 1908, which may include iliac arteries or the like. The distal branch vessels 1908 may extend radially and distally from the diseased section 1906 of the diseased blood vessel 1902. The diseased blood vessel 1902, the branch vessels 1904, and the distal branch vessels 1908 may be defined by blood vessel walls 1910.

In FIG. 19A, a deployment guidewire 1912 and optional guidewires 502 are positioned extending through the patient 1920. The guidewires 502 and the deployment guidewire 1912 may be advanced from an insertion site 1922 (e.g., a femoral, jugular, or other insertion site), past a treatment site (e.g., the diseased section 1906 of the diseased blood vessel 1902), out through an extraction site 1926 (e.g., a femoral, jugular, or other extraction site). In some examples, an extraction sheath, an insertion sheath, an extraction snare, and/or an insertion snare may be advanced through the insertion site 1922 and/or the extraction site 1926 in order to aid in advancing the guidewires 502 and the deployment guidewire 1912 through the insertion site 1922, past the treatment site, and out through the extraction site 1926. In some examples, the insertion site 1922 may be a femoral insertion site, as illustrated in FIG. 19A, the guidewires 502 and the deployment guidewire 1912 may pass through one of the branch vessels 1904, and the extraction site 1926 may be a jugular extraction site.

The guidewires 502 may extend through the fenestrated endovascular prosthesis 100 and the deployment device 800. For example, the guidewires 502 may extend into a delivery catheter assembly 804 of the deployment device 800 adjacent a distal tip 806. The guidewires 502 may extend through a bore 106 of the fenestrated endovascular prosthesis 100 in a proximal portion 112 of the fenestrated endovascular prosthesis 100, through fenestration tubes 104, and outside the fenestrated endovascular prosthesis 100 in a distal portion 114 of the fenestrated endovascular prosthesis 100. The guidewires 502 may extend through guide lumens 1410 in an intermediate sheath 932 of the delivery catheter assembly 804, through a handle assembly 802 of the deployment device 800, and out of the handle assembly 802 adjacent a luer fitting 936 of the deployment device 800. The deployment guidewire 1912 may extend into the distal tip 806 of the deployment device 800. The deployment guidewire 1912 may extend through a lumen 940 of an inner sheath 930 of the deployment device 800 and out of the luer fitting 936.

FIGS. 19B through 19E illustrate detail views of the diseased blood vessel 1902, and specifically, deployment of the fenestrated endovascular prosthesis 100 in the diseased blood vessel 1902. In FIG. 19B, the guidewires 502 and the deployment guidewire 1912 are positioned through the diseased blood vessel 1902. The fenestrated endovascular prosthesis 100 and the guidewires 502 can be advanced relative to the deployment guidewire 1912. The fenestrated endovascular prosthesis 100 is partially deployed in the diseased blood vessel 1902 in FIG. 19B. The deployment guidewire 1912 may be formed of materials similar to or the same as the guidewires 502 and may have similar or the same characteristics as the guidewires 502 (e.g., diameters and the like).

FIG. 19B illustrates four of the guidewires 502 extending through the delivery catheter assembly 804 and the fenestrated endovascular prosthesis 100. However, any number of the guidewires 502, in any desired configuration, may be provided in the delivery catheter assembly 804 and the fenestrated endovascular prosthesis 100. Further, all four of the guidewires 502 are illustrated as extending through the fenestrated endovascular prosthesis 100. However, in some examples, the guidewires 502 may extend partially through the fenestrated endovascular prosthesis 100. For example, the guidewires 502 may extend partially through the fenestration tubes 104 of the fenestrated endovascular prosthesis 100, through the fenestration tubes 104 and partially through the proximal portion 112 of the fenestrated endovascular prosthesis 100, or the like. In some examples, the guidewires 502 may be positioned in the fenestrated endovascular prosthesis 100 after the fenestrated endovascular prosthesis 100 is deployed. In some examples, radiopaque marker bands 214 can be disposed adjacent distal openings 212 of the fenestration tubes 104 to facilitate access to the fenestration tubes 104 with the guidewires 502 using fluoroscopy. Providing the guidewires 502 extending completely through the fenestrated endovascular prosthesis 100 may reduce the likelihood of the guidewires 502 being dislodged or otherwise removed from the fenestration tubes 104 of the fenestrated endovascular prosthesis 100. In examples in which the guidewires 502 extend partially through the fenestrated endovascular prosthesis 100, the fenestration tubes 104 can be opened and secondary endovascular prostheses can be deployed from one direction, such as from the insertion site 1922.

The delivery catheter assembly 804 may be advanced along the deployment guidewire 1912 from the insertion site 1922 to the treatment site (e.g., the diseased section 1906 of the diseased blood vessel 1902). The guidewires 502 can be advanced along with the delivery catheter assembly 804, such that the guidewires 502 remain at the same position relative to the delivery catheter assembly 804 and the fenestrated endovascular prosthesis 100. The relative position and orientation of the delivery catheter assembly 804 relative to the diseased section 1906 of the diseased blood vessel 1902 may be verified using radiographic imaging or the like. Specifically, radiographic imaging may be used to detect radiopaque marker bands 120 on the fenestrated endovascular prosthesis 100, as well as radiopaque marker bands 1504 and orientation indicia (such as the orientation indicium 1502a or the orientation indicium 1502b) on the distal tip 806.

Once the delivery catheter assembly 804 and the fenestrated endovascular prosthesis 100 are positioned in a desired location and orientation, the handle assembly 802 of the deployment device 800 may be used to deploy the fenestrated endovascular prosthesis 100. For example, an actuator 904 may be displaced in order to retract an outer sheath 928 of the delivery catheter assembly 804 relative to the inner sheath 930 and the intermediate sheath 932 of the delivery catheter assembly 804, exposing the fenestrated endovascular prosthesis 100. As the fenestrated endovascular prosthesis 100 is exposed, the fenestrated endovascular prosthesis 100 can self-expand.

In FIG. 19C, the fenestrated endovascular prosthesis 100 is fully deployed in the diseased blood vessel 1902 and the deployment guidewire 1912 and the delivery catheter assembly 804 are removed from the diseased blood vessel 1902. The fenestrated endovascular prosthesis 100 is fully deployed in the diseased blood vessel 1902 once the outer sheath 928 is retracted from the length of the fenestrated endovascular prosthesis 100. As illustrated in FIG. 19B, the fenestrated endovascular prosthesis 100 can be deployed from a proximal end 108 to a distal end 110 of the fenestrated endovascular prosthesis 100. A spring force from a wire stent 122 of the fenestrated endovascular prosthesis 100 exerts outward radial force from the fenestrated endovascular prosthesis 100 to the blood vessel walls 1910, which seals the fenestrated endovascular prosthesis 100 to the blood vessel walls 1910 of the diseased blood vessel 1902. The proximal portion 112 of the fenestrated endovascular prosthesis 100 can be sealed to the blood vessel walls 1910 above the diseased section 1906, which may occur when the fenestrated endovascular prosthesis 100 is partially deployed. The distal portion 114 of the fenestrated endovascular prosthesis 100 can be sealed to the blood vessel walls 1910 below the diseased section 1906, which may occur once the fenestrated endovascular prosthesis 100 is fully deployed. This allows the fenestrated endovascular prosthesis 100 to bypass the diseased section 1906. The radial stress between the fenestrated endovascular prosthesis 100 and the blood vessel walls 1910 also secures the fenestrated endovascular prosthesis 100 relative to the blood vessel walls 1910.

Upon initial deployment of the fenestrated endovascular prosthesis 100, fenestration tubes 104 of the fenestrated endovascular prosthesis 100 may be in the sealed configuration. As such, blood may flow through the bore 106 of the fenestrated endovascular prosthesis 100, without flowing through the fenestration tubes 104. While three fenestration tubes 104 are illustrated in FIG. 19C, the steps of deploying and implanting the fenestrated endovascular prosthesis 100 can be applied to fenestrated endovascular prostheses 100 having more or fewer fenestration tubes 104.

In FIG. 19D, selected fenestration tubes 104 are un-sealed from the sealed configuration to the open configuration. The guidewires 502 can be used to transition the fenestration tubes 104 from the sealed configuration to the open configuration. For example, a practitioner may identify particular guidewires 502 that extend through fenestration tubes 104 that are to be un-sealed to the open configuration. The practitioner may identify particular guidewires 502 extending through selected fenestration tubes 104 by observing identification features (such as the identification features 1802a, the identification features 1802b, or the identification features 1802c, discussed above with respect to FIGS. 18A through 18C) disposed at either end of each of the guidewires 502. For example, per an instruction for use (IFU) a first guidewire 502 disposed in a first fenestration tube 104 may have one kink, bend, or nub; a second guidewire 502 disposed in a second fenestration tube 104 may have two kinks, bends, or nubs; and a third guidewire 502 disposed in a third fenestration tube 104 may have three kinks, bends, or nubs. In the example illustrated in FIG. 19D, two of the guidewires 502 are used to un-seal two of the fenestration tubes 104 from the sealed configuration to the open configuration.

As illustrated in FIG. 19D, fenestration dilators 1914 can be advanced along the guidewires 502 to un-seal the fenestration tubes 104 from the sealed configuration to the open configuration. The fenestration dilators 1914 may be similar to or the same as the fenestration dilators discussed above (e.g., the fenestration dilator 1600a, the fenestration dilator 1600b, the fenestration balloon dilator 1700a or the fenestration balloon dilator 1700b, discussed above with respect to FIGS. 16A through 17B). The fenestration dilators 1914 may be advanced through seal regions 216 (illustrated in FIG. 19C) in each of the selected fenestration tubes 104 to un-seal the seal regions 216. In the example illustrated in FIG. 19D, the fenestration dilators 1914 may be advanced from the insertion site 1922 (e.g., a femoral insertion site or the like); however, in some examples, the fenestration dilators 1914 may be advanced from the extraction site 1926 (e.g., a jugular insertion site or the like). In some examples, the fenestration tubes 104 can act as funnels to funnel the fenestration dilators 1914 towards the seal regions 216 in the fenestration tubes 104.

When a fenestration tube 104 is in the open configuration, blood is allowed to flow from a proximal opening 210, through the fenestration tube 104, and out of a distal opening 212 of the fenestration tube 104, such as into the diseased section 1906. Thus, only selected ones of the fenestration tubes 104 may be un-sealed, while un-selected ones of the fenestration tubes 104 remain in the sealed configuration with the seal regions 216 intact. Thus, each of the fenestration tubes 104 can be selectively un-sealed for use, or left in a sealed configuration by a practitioner during treatment. This allows for the fenestrated endovascular prosthesis 100 to be configurable based on specific patients, and reduces variations of the fenestrated endovascular prosthesis 100 needed to treat specific patients. The guidewires 502 may be removed from un-selected ones of the fenestration tubes 104 with the seal regions 216 intact, such that blood flow through the un-selected fenestration tubes 104 is prevented. In some examples, the fenestrated endovascular prosthesis 100 may be deployed in a vessel without branched arteries or vessels, each of the fenestration tubes 104 can be left in the sealed configuration such that the fenestrated endovascular prosthesis 100 functions as a non-fenestrated endovascular prosthesis.

In FIG. 19E, secondary endovascular prostheses 702 are sealingly deployed in selected ones of the fenestration tubes 104. As illustrated in FIG. 19E, the secondary endovascular prostheses 702 may be deployed such that proximal ends 704 of the secondary endovascular prostheses 702 are proximal to proximal openings 210 of the fenestration tubes 104. This provides for maximal sealing between the secondary endovascular prostheses 702 and the respective fenestration tubes 104. In some examples, the proximal ends 704 of the secondary endovascular prostheses 702 can be positioned distal to or at a same level as the proximal openings 210 of the fenestration tubes 104. The secondary endovascular prostheses 702 are further sealingly deployed in the branch vessels 1904. The secondary endovascular prostheses 702 thus provide flow paths for blood from the bore 106 of the fenestrated endovascular prosthesis 100 through the secondary endovascular prostheses 702 and the fenestration tubes 104 to the branch vessels 1904.

The secondary endovascular prostheses 702 may be deployed by exchanging the guidewires 502 with steerable guidewires or similar elongate medical instruments. As an example, a guidewire 502 may be used to pull a steerable guidewire from the extraction site 1926 (e.g., a proximal access site, such as a jugular access site) through a respective fenestration tube 104. The steerable guidewire can then be disconnected from the guidewire 502 and the guidewire 502 can be removed from the patient's body, such as through the insertion site 1922 (e.g., a distal access site, such as a femoral access site). The steerable guidewire can be steered into a respective branch vessel 1904. Once the steerable guidewire is positioned in the respective branch vessel 1904, a secondary endovascular prosthesis 702 can be advanced along the steerable guidewire from the extraction site 1926. The secondary endovascular prosthesis 702 may be advanced along the steerable guidewire by a secondary deployment device, which may be similar to the deployment device 800. The secondary endovascular prosthesis 702 can be retained in the secondary deployment device in a crimped or delivery configuration and deployed to a deployed configuration similar to the fenestrated endovascular prosthesis 100. As illustrated in FIG. 19E, the secondary endovascular prosthesis 702 can be deployed to provide a bridge between the fenestration tube 104 of the fenestrated endovascular prosthesis 100 and the branch vessel 1904.

The secondary endovascular prostheses 702 can be deployed using any suitable secondary deployment devices. The secondary endovascular prostheses 702 can be configured to be expanded by a balloon, or can include wire stents and be self-expanding similar to the fenestrated endovascular prosthesis 100.

Providing a fenestrated endovascular prosthesis 100 that includes fenestration tubes 104 allows the fenestrated endovascular prosthesis 100 to be deployed to repair arteries, blood vessels, and the like, even in cases where branch vessels extend from damaged sections of the arteries. The fenestrated endovascular prosthesis 100 may be deployed with guidewires 502 extending through the fenestration tubes 104, which aids a practitioner in locating the fenestration tubes 104 for additional treatments, such as deploying secondary endovascular prostheses 702 between the fenestration tubes 104 and branch vessels adjacent damaged sections of the arteries repaired by the fenestrated endovascular prosthesis 100. Identification features may be provided on the guidewires 502 to aid the practitioner in selecting a particular guidewire 502 that extends through a selected fenestration tube 104 in which a secondary endovascular prosthesis 702 is to be deployed. A deployment device 800 may include various channels through which the guidewires 502 slidably extend, which allows the fenestrated endovascular prosthesis 100 to be deployed with the guidewires 502 already being positioned through the fenestration tubes 104.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A method of implanting a fenestrated endovascular prosthesis, the method comprising:
providing a fenestrated endovascular prosthesis comprising a guidewire extending through a fenestration;
deploying the fenestrated endovascular prosthesis at a treatment site within a blood vessel; and
un-sealing a seal region of a lumen in communication with the fenestration to transition the lumen from a sealed configuration to an open configuration, wherein:
after un-sealing the seal region, the lumen is in fluid communication with a bore of the fenestrated endovascular prosthesis and an exterior of the fenestrated endovascular prosthesis.

2. The method of claim 1, further comprising deploying a secondary endovascular prosthesis within the lumen and within a branch vessel of the blood vessel, wherein the secondary endovascular prosthesis is in fluid communication with the bore of the endovascular prosthesis and with the branch vessel.

3. The method of claim 2, further comprising replacing the guidewire with a steerable guidewire, wherein the steerable guidewire is advanced into the branch vessel, and wherein the secondary endovascular prosthesis is deployed after advancing the secondary endovascular prosthesis along the steerable guidewire.

4. The method of claim 1, wherein un-sealing the seal region comprises advancing a tapered fenestration dilator along the guidewire into the seal region.

5. The method of claim 1, further comprising radially expanding the secondary endovascular prosthesis to form a seal between an exterior surface of the secondary endovascular prosthesis and an interior surface of the lumen.

6. The method of claim 2, wherein the secondary endovascular prosthesis is positioned with a proximal end of the secondary endovascular prosthesis disposed proximal to a proximal end of the lumen and a distal end of the secondary endovascular prosthesis disposed in the branch vessel prior to the secondary endovascular prosthesis being deployed.

7. The method of claim 2, wherein un-sealing the seal region comprises:
advancing a fenestration balloon dilator along the guidewire into the seal region; and
expanding the fenestration balloon dilator to un-seal the seal region.

8. A method of bypassing a diseased segment of a vessel, the method comprising:
deploying a primary endovascular prosthesis within a primary vessel, wherein:
a proximal portion of the primary endovascular prosthesis sealingly engages a non-diseased portion of the primary vessel;
blood flow to a first branch vessel of the primary vessel is restricted by the primary endovascular prosthesis;
a first guidewire extends through a first fenestration disposed in a wall of the primary endovascular prosthesis; and
a second guidewire extends through a second fenestration disposed in the wall of the primary endovascular prosthesis;
opening a seal region of a lumen of the first fenestration such that the lumen is in fluid communication with a bore of the primary endovascular prosthesis,
deploying a secondary endovascular prosthesis in the lumen such that the secondary endovascular prosthesis is in fluid communication with the bore of the primary endovascular prosthesis and the first branch vessel.

9. The method of claim 8, wherein the seal region is opened by advancing a dilator along the first guidewire from a distal end of the first guidewire.

10. The method of claim 9, wherein the secondary endovascular prosthesis is advanced along the first guidewire from a proximal end of the first guidewire.

11. The method of claim 9, wherein:
the first guidewire is used to advance a steerable guidewire from a proximal access site through the lumen;
the method further comprises steering the steerable guidewire into the first branch vessel; and
the secondary endovascular prosthesis is advanced along the steerable guidewire from a proximal end of the steerable guidewire.

12. The method of claim 8, wherein the secondary endovascular prosthesis is positioned extending from proximal a proximal end of the first fenestration distally into the first branch vessel.

13. The method of claim 8, further comprising radially expanding the secondary endovascular prosthesis to form a seal between an exterior surface of the secondary endovascular prosthesis and an interior surface of the lumen.

14. A method comprising:
advancing a guidewire from an insertion site, through a treatment site, to an extraction site; and
advancing a fenestrated endovascular prosthesis to the treatment site, wherein:
the guidewire extends through a fenestration tube of the fenestrated endovascular prosthesis between a bore of the fenestrated endovascular prosthesis and an exterior of the fenestrated endovascular prosthesis; and
the fenestration tube comprises a seal region encircling the guidewire when the fenestration tube is in a sealed configuration.

15. The method of claim 14, further comprising advancing a dilator along the guidewire to un-seal the seal region such that the fenestration tube is in an open configuration.

16. The method of claim 15, further comprising deploying a secondary endovascular prosthesis in the fenestration tube after the fenestration tube is in the open configuration.

17. The method of claim 14, further comprising:
advancing a steerable instrument through the fenestration tube into a branch vessel adjacent the treatment site;
advancing a secondary endovascular prosthesis along the steerable instrument into the fenestration tube and the branch vessel; and
deploying the secondary endovascular prosthesis such that the secondary endovascular prosthesis is sealed to the fenestration tube and the branch vessel.

18. The method of claim 14, further comprising:
unsealing the fenestration tube to an open configuration;
advancing a secondary endovascular prosthesis into the fenestration tube in the open configuration such that a proximal end of the secondary endovascular prosthesis extends into the bore of the fenestrated endovascular prosthesis proximal to a proximal end of the fenestration tube; and
sealingly deploying the secondary endovascular prosthesis in the fenestration tube.

19. The method of claim 18, further comprising determining relative positions of the secondary endovascular prosthesis and the fenestration tube using radioscopy prior to sealingly deploying the secondary endovascular prosthesis.

20. The method of claim 14, further comprising removing the guidewire from the fenestration tube, wherein blood flow out of the fenestrated endovascular prosthesis through the fenestration tube is restricted by the seal region after the guidewire is removed.
